(19) United States
(12) Patent Application Publication
(10) Pub. No.: US 2016/0000842 A1
(43) Pub. Date: Jan. 7, 2016

(54) ONCOLYTIC VIRUS

(71) Applicant: BAYLOR COLLEGE OF MEDICINE, Houston, TX (US)

(72) Inventors: Xiao-Tong Song, Pearland, TX (US); Stephen M. G. Gottschalk, Houston, TX (US); Feng Yu, Houston, TX (US)

(21) Appl. No.: 14/770,061

(22) PCT Filed: Mar. 5, 2014
(86) PCT No.: PCT/US14/20935

§ 371 Date: Aug. 24, 2015

Related U.S. Application Data
(60) Provisional application No. 61/772,803, filed on Mar. 5, 2013.

(51) Int. Cl. A61K 35/768; A61K 45/06; C07K 16/32; C12N 7/00; C07K 16/28

(52) U.S. Cl. CPC A61K 35/768; C12N 7/00; C07K 16/2803; C07K 16/32; A61K 45/06; C12N 2710/24121; C12N 2710/24132; C07K 2317/622; C07K 2317/31; C12N 2710/24143

(57) ABSTRACT

Embodiments of the present disclosure concern oncolytic viruses, such as vaccinia virus, for example, for the treatment of cancer, wherein the viruses encode an engager molecule having an activation domain that recognizes a cell molecule, such as CD3, for example, on T cells and an antigen recognition domain that recognizes a tumor antigen, such as EphA2, HER2, GD2, or Glypican-3, for example. In some embodiments, the engager molecules further comprise a cytokine or co-stimulatory domain, for example. Methods of treating cancer using one or more of the compositions are encompassed in the disclosure.

This application claims priority to U.S. Provisional Patent Application Ser. No. 61/772,803, filed Mar. 5, 2013, which is incorporated by reference herein in its entirety.

### TECHNICAL FIELD

The fields of the disclosure include at least immunology, virology, cell biology, molecular biology, and medicine, including cancer medicine.

### BACKGROUND

While cure rates for several malignancies have significantly improved, the outcome for patients with advanced solid tumors remains grimly unchanged over the last decades, underscoring the need for new therapies. Oncolytic vaccinia virus is a useful addition to the current treatment options of solid tumors because of its safety and its ability to infect, replicate in, and lyse tumor cells. Although clinical studies have shown that intratumoral or intravenous injection of oncolytic vaccinia virus are safe and can induce tumor lysis, the antitumor efficacy of the oncolytic vaccinia virus is suboptimal and most tumors occurred, highlighting the need for further improvement of oncolytic vaccinia virus therapy.

### BRIEF SUMMARY

The present disclosure is directed to methods and/or compositions for immunotherapy for an individual. In specific embodiments the individual is a mammal that has cancer and is in need of cancer therapy. The cancer may be of any kind, including lung, breast, prostate, pancreatic, liver, colon, stomach, spleen, skin, brain, blood, kidney, thyroid, and so forth. The cancer may be solid tumors. In particular cases, the cancer has one or more tumor markers, including tumor antigens. One or more tumor antigens may be present in at least some of the cancer cells, and the one or more tumor antigens may be the target of the immunotherapy, in particular aspects of the disclosure.

In particular embodiments of the disclosure, provided herein are methods and compositions related to recombinant oncolytic viral vectors suitable for use in immunotherapy, e.g., T cell engager-armed oncolytic viruses (TEA-OVs). The TEA-OV provided herein provide an advantage over oncolytic viruses, alone, and T cells, alone, in that the recombinant oncolytic virus, in addition to killing tumor cells directly, stimulates T cells within the vicinity of the tumor being infected by the virus to kill tumor cells, as well. In specific embodiments, the vectors are intratumorally injected or injected intravenously, although other means of delivery are encompassed in the disclosure. In certain embodiments, the vector comprises bi-specificity activity, and in particular embodiments it concerns the presence of an activation domain and an antigen recognition domain. In particular aspects, the oncolytic viral vector is a T cell engager-armed vaccinia virus, termed TEA-VV.

In a first aspect, provided herein are recombinant oncolytic viruses, such as oncolytic vaccinia viruses, for example, that have been engineered to comprise an expression region with a promoter directing expression of a nucleic acid encoding a bi-specific T cell engager polypeptide, such as, for example, an engager that targets EphA2, HER2, GD2 or Glypican-3. The bi-specific T-cell engager polypeptide, in particular embodiments, comprises an activation domain and an antigen recognition domain. In certain embodiments, the activation domain is a receptor or ligand for a T cell receptor polypeptide, e.g., a polypeptide that binds to CD3 so as to activate the T cell. In certain embodiments, the antigen recognition domain is a receptor or a ligand for a cell surface protein on a target cell, e.g., a cancer cell. In more specific embodiments, one or both of the activation domain and/or antigen recognition domain is an antibody, e.g., a single-chain antibody, e.g., a single-chain variable fragment (scFv). In specific embodiments, the antigen recognition domain binds to molecules that are present on target cells, and the activation domain binds to cell receptors that elicit processes that ultimately are toxic to the recipient target cell. In specific embodiments, bi-specificity relates to two antibody fragments or antigen binding fragment or derivative thereof, such as single chain variable fragments (scFvs). Although in certain embodiments one scFv is specific for CD3, in alternative embodiments, the scFv is specific for another component of the TCR complex. The skilled artisan recognizes that the TCR complex is an octomeric complex of variable TCR α and β chains with three dimeric signaling modules CD3δ/ε, CD3γ/ε and CD3 ζ/ζ or ζ/η. Although in some cases the compositions disclosed herein target CD3ε with an scFV, targeting other CD3 molecules, especially CD3ζ, or the TCR α and β chains, with a specific scFv is encompassed in the disclosure. In specific embodiments, targeting molecules that are not part of the TCR complex (CD27, CD28, CD40, CD134, CD137, and CD278) is encompassed in the disclosure. In specific embodiments one scFv is specific for CD3 molecule on T lymphocytes and the other scFv is specific for a particular tumor antigen of choice.

In certain embodiments, and not to be bound by theory, a T cell engager-armed oncolytic virus, e.g., a T cell engager-armed oncolytic vaccinia virus, facilitates T cell infiltration in tumors and, through the virally-expressed T cell engager, induces bystander killing of tumor cells that are not infected by the virus, leading to an enhanced antitumor effect.

The particular scFv for the tumor antigen may be selected or tailored to recognize a corresponding cancer cell comprising, e.g. displaying on its cell surface, the particular tumor antigen. In particular embodiments, the tumor antigen is EphA2, HER2, GD2, Glypican-3, 5T4, 8H9, αᵥβ₆ integrin, B7-H3, B7-H6, CAIX, CA9, CD19, CD20, CD22, kappa light chain, CD30, CD33, CD38, CD44, CD44v6, CD44v7/8, CD70, CD123, CD138, CD171, CEA, CSPG4, EGFR, EGFRvIII, EGP2, EGP40, EPCAM, ERBB3, ERBB4, ErbB3/4, FAP, FAR, FBP, fetal AchR, Folate Receptor α, GD2, GD3, HLA-AI MAGE A1, HLA-A2, IL11Ra, IL13Ra2, KDR, Lambda, Lewis-Y, MCSP, Mesothelin, Muc1, Muc16, NCAM, NKG2D ligands, NY-ESO-1, PRAME, PSCA, PSC1, PSMA, ROR1, SURVIVIN, TAG72, TEM1, TEM8, VEGRR2, carcinoembryonic antigen, HMW-MAA, VEGF receptors, and other exemplary antigens are antigens that are present with in the extracellular matrix of tumors, such as oncofetal variants of fibronectin, tenascin, or necrotic regions of tumors.

In a specific embodiment, provided herein is a oncolytic vaccinia virus comprising a polynucleotide that encodes a bi-specific T cell engager molecule that targets EphA2 and induces lysis of EphA2-expressing tumors more effectively than an un-modified vaccinia virus. In specific embodiments, the viral vector encodes a bi-specific T cell engager molecule and also encodes one or more polynucleotides that express co-stimulatory molecules, including CD70, CD80, CD83, CD86, CD134L (OX40L), and CD137L (41BBL). In some embodiments, the viral vector encodes at least one dimerization domain or at least one trimerization domain. The dimerization or trimerization domain may be positioned on a viral vector in certain configuration. In specific embodiments, the nucleic acid sequence that encodes the dimerization or trimerization domain is positioned between the nucleic acid that encodes the activation domain and the antigen recognition domain. Particular exemplary compositions of the disclosure comprise the sequence of SEQ ID NO:1, SEQ ID NO:2, SEQ ID NO:3, SEQ ID NO:4, SEQ ID NO:5, SEQ ID NO:6, SEQ ID NO:7, for example.

The vaccinia virus may be the Wyeth, Modified Vaccinia. Ankara (MVA), Lister, or Western Reserve (WR) strain, in certain embodiments. The promoter may be a vaccinia virus promoter, a synthetic promoter, a promoter that directs transcription during at least the early phase of infection, or a promoter that directs transcription during at least the late phase of infection.

An exemplary TEA-VV of the disclosure expresses the functional bi-specific engager molecule in vitro and in vivo; displays similar virus/replication/tumor lysis efficacy compared to unmodified vaccinia virus; induces tumor killing more effectively in the presence of human T cells, compared to unmodified vaccinia virus; and/or induces bystander killing of cells that are not infected with virus effectively; the composition also inhibits tumor progression in vivo. Merely as a demonstrative example, A549 adenocarcinomic human alveolar basal epithelial cells are killed with methods of the disclosure.

In another embodiment, any type of suitable oncolytic viral vector other than oncolytic vaccinia virus may be employed to express bi-specific T cell engager molecules for cancer therapy. In specific embodiments the vector is an oncolytic adenoviral vector (AdV), herpes simplex virus (HSV), reovirus, myxoma virus (MYXV), poliovirus, vesicular stomatitis virus (VSV), measles virus (MV), and Newcastle disease virus (NDV), and so forth). In certain embodiments, the viral polynucleotide encodes a fusion molecule comprising an activation domain and an antigen recognition domain, and in specific embodiments each domain is a scFv. The activation domain may be positioned toward the N-terminus of the polypeptide in relation to the antigen recognition domain, or the activation domain may be positioned toward the C-terminus of the polypeptide in relation to the antigen recognition domain.

The recombinant oncolytic viruses may be generated by any suitable genetic recombination method in the art.

In another aspect, provided herein is a method of treating an individual having cancer comprising administering to the individual a therapeutically effective amount of a TEA-OV, e.g., wherein said TEA-OV expresses a T cell engager that comprises an antigen recognition domain that binds to a TAA or TSA displayed by said cancer. In particular embodiments, the individual is administered recombinant T cell engager-armed vaccinia virus (TEA-VV). In specific embodiments, the TEA-VV is administered intratumorally, intravascularly, intravenously, intraarterially, intraperitoneally, subcutaneously, intrathecally, or intranasally.

In certain embodiments, provided herein is a method of treating an individual for cancer, comprising the step of delivering to the individual a therapeutically effective amount of a virus of the disclosure, including an oncolytic virus that encodes a bispecific molecule comprising a scFv specific for a cell surface molecule and a scFv specific for a tumor antigen. In particular embodiments, the amount of the virus administered to the individual, e.g., the therapeutically effective amount, comprises 10⁵-10¹³ pfu of the virus. Methods of the disclosure may include the step of delivering to the individual an additional cancer therapy, such as surgery, radiation, chemotherapy, immunotherapy, hormone therapy, or a combination thereof. Methods of the disclosure may include the step of identifying that an individual is in need of cancer therapy.

In one embodiment, there is a genetically engineered oncolytic virus comprising a nucleic acid sequence that encodes a molecule comprising an activation domain that binds to a target on an immune cell and an antigen recognition domain that binds one or more molecules produced by or present on a target cell. In specific embodiments, said activation domain and said antigen recognition domain are attached by a linker. In some embodiments, the virus further comprises nucleic acid that encodes one or more co-stimulatory molecules. In certain embodiments, the virus further comprises nucleic acid that encodes a dimerization domain. In particular embodiments, the virus further comprises nucleic acid that encodes a trimerization domain. In some embodiments, the activation domain comprises an antibody, ligand, receptor, or peptide. In certain embodiments, the immune cell is a T-cell and the activation domain is an antibody that recognizes CD3, and in some embodiments the immune cell is a NK cell and the activation domain is an antibody recognizes CD16, NKG2D, or NKp30. In some embodiments, the antibody is a single chain fragment variable (scFv) antibody. In certain embodiments, the antigen recognition domain is an antibody that recognizes an antigen produced by target cells. In particular cases, the antibody is a scFv antibody. In some embodiments, the antigen recognition domain is a ligand, a peptide, or a soluble TCR. In particular embodiments, the virus encodes a polypeptide that comprises the sequence of SEQ ID NO:1, SEQ ID NO:2, SEQ ID NO:3, SEQ ID NO:4, SEQ ID NO:5, SEQ ID NO:6, or SEQ ID NO:7. Embodiments also include a virus wherein the antigen is a tumor antigen, such as one selected from the group consisting of EphA2, HER2, GD2, Glypican-3, 5T4, 8H9, αᵥβ₆ integrin, B7-H3, B7-H6, CAIX, CD19, CD20, CD22, CD30, CD33, CD44, CD44v6, CD44v7/8, CD70, CD123, CD138, CD171, CEA, CSPG4, EGFR, EGFRvIII, EGP2, EGP40, EPCAM, ERBB3, ERBB4, FAP, FAR, FBP, fetal AchR, FRα, GD3, HLA-A1+MAGE1, IL11Rα, IL13Rα2, Kappa, KDR, Lambda, Lewis-Y, MCSP, Mesothelin, Muc1, Muc16, NCAM, NKG2D ligands, NY-ESO-1, PRAME, PSCA, PSMA, ROR1, SURVIVIN, TAG72, TEM1, TEM8, and VEGRR2. In certain embodiments, the target on an immune cell is selected from the group consisting of CD3, CD16, NKG2D, NKp30, and an invariant TCR. The target on an immune cell may be CD3. In some cases, the oncolytic virus is vaccinia virus, although in some embodiments the oncolytic virus is adenovirus, Herpes simplex virus 1 (HSV1), myxoma virus, reovirus, poliovirus, vesicular stomatitis virus (VSV), measles virus (MV), or Newcastle disease virus (NDV). In specific embodiments, the one or more co-stimulatory molecules is selected from the group consisting of 4-1BBL, CD80, CD86, CD83, and OX40L. In some embodiments, the dimerization domain is IgG1 CH2CH3 domain, Leucine zipper, helix-loop-helix, ankyrin, or PAS domain. In particular embodiments, the trimerization domain is from human collagen type XV NCI, collagen type I, collagen type II, collagen type III, collagen type IV, collagen type V, collagen type XI, C-terminal domain of T4 fibritin, or Nemo trimerization domain.

In an embodiment of the disclosure, there is provided a method of treating an individual for cancer, comprising the step of delivering to the individual a therapeutically effective amount of a virus of the disclosure. In some embodiments, the amount of the virus comprises 10⁵-10¹³ pfu of the virus. In some embodiments, the method further comprises the step of delivering to the individual an additional cancer therapy, such as surgery, radiation, chemotherapy, immunotherapy, hormone therapy, or a combination thereof. In some embodiments, the method further comprises the step of identifying that the individual is in need for cancer treatment.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 shows an exemplary rationale for TEA-VV;

FIG. 2 provides an exemplary scheme of expression cassettes encoding EphA2-scFv-CD3-scFv (EphA2-TEA-VV) or GFP (GFP-VV);

FIG. 3 shows that viral replication is required for GFP expression under the transcriptional control of F17R late promoter;

FIG. 4 shows that EphA2-TEA-VV expressed secretory bi-specific EphA2-scFv-CD3-scFv with the ability of binding both to human T cells and tumor antigen EphA2. A549 tumor cells (1×10⁶/2 ml/well in 6 well plate) were infected with EphA2-TEA-VV or GFP-VV at MOI 5. The culture medium were collected after 24 hours incubation and added to 1×10⁶ unstimulated PBMC, followed by staining of EphA2-FITC, CD8-PE and CD4-APC. The inserted numbers were the percentage of EphA2 positive cell population from gated CD8+ or CD4+ cells;

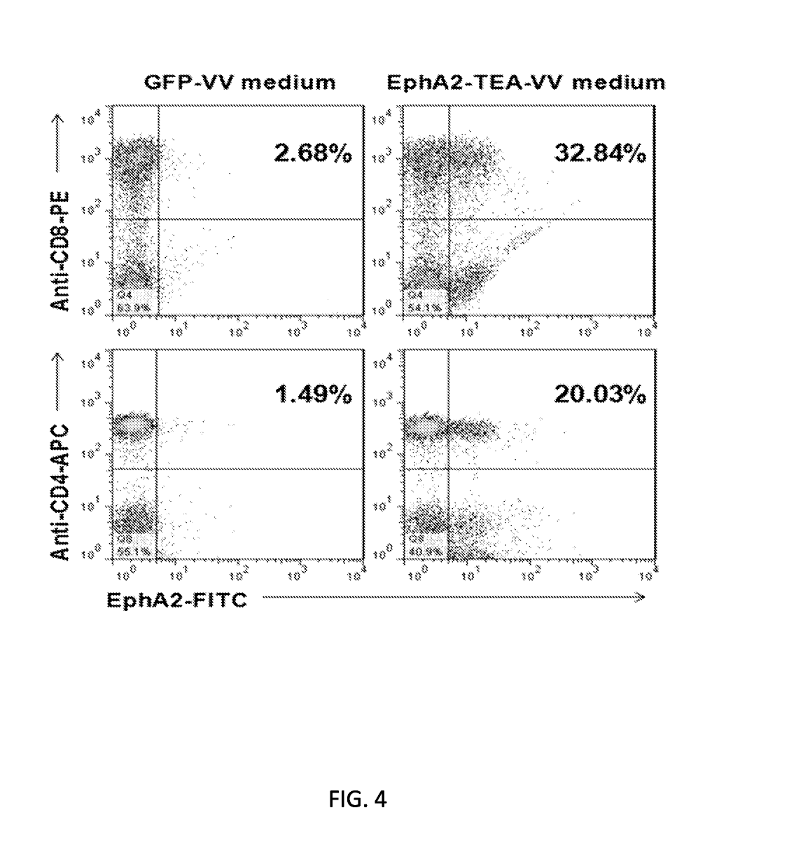

FIGS. 5A and 5B show that EphA2-TEA-VV displayed similar viral replication ability, compared to parent VV, in CV-1 cells and normal human cells. The replication of EphA2-TEA-VV, GFP-VV, and vSC20 in CV-1 (A) and normal human fibroblast (B) are presented. Cells were infected at a MOI of 0.1 and after 1, 2, or 3 days, viral titers were determined by plaque assays in CV-1 cells;

FIG. 6 demonstrates that EphA2-TEA-VV displayed similar tumor lytic ability against EphA2+A549 tumor cells, compared to GFP-VV, in the absence of human T cells. A549 tumor cells were infected with increasing doses (MOI 0.01, 0.1, 1, or 5) of EphA2-TEA-VV (EphA2-VV), or GFP-VV. The cell viability at 48 hours post infection was determined by MTS assays. The results showed that EphA2-TEA-VV, or GFP-VV exhibited similar tumor lytic activity against A549 tumor cells in the absence of human T cells.

FIG. 7 demonstrates that EphA2-TEA-VV displayed enhanced tumor lytic ability against EphA2+A549 tumor cells in the presence of human T cells, compared to GFP-VV.A549 tumor cells were infected with EphA2-TEA-VV or GFP-VV at MOI 0.1. In some experiment, human T cells were co-cultured with A549 cells (T:A549=5:1). The cell viability at various time points post infection was determined by MTS assays;

FIGS. 8A and 8B demonstrate that tumor lytic ability of the oncolytic EphA2-TEA-VVs against EphA2+A549 tumor cells in the presence of human T cells is virus dose dependent. A549 tumor cells were infected with EphA2-TEA-VV with increasing doses (MOI 0.001, 0.01, 0.1, or 1). In some experiments, human T cells were co-cultured with A549 cells (T:A549=5:1). The cell viability at 24 or 48 hr post infection was determined by MTS assays;

FIGS. 9A-9D demonstrate that EphA2-TEA-VV activated human T cells. A549 cells were infected with EphA2-TEA-VV or GFP-VV at MOI 0.1 or 1. Infected A549 cells were either cultured in the presence of human T cells (T cell: A549 ration=5:1). 24, 48, or 72 hours later, supernatants were collected and IFN-γ (A & B) and IL-2 (C & D) production was determined by ELISA;

FIG. 10 demonstrates that EphA2-TEA-VV induced by-stander killing of tumor cells. A549 were infected with EphA2-TEA-VV or GFP-VV at various MOI and after 24 hours, supernatants were collected and used in a co-culture assay of PBMCs and A549 cells (PBMC:tumor cells=5:1). After 48 hours, tumor cell killing was measured by MTS assay;

FIG. 11 shows that EphA2-TEA-VV resulted in an enhanced antitumor response in A549 s.c. tumor model. 2×10⁶ A549 cells were mixed 1×10⁷ unstimulated PBMC and inoculated s.c. into the right flank of SCID mice on day 0, immediately followed by i.p. injection of 1×10⁸ PFU EphA2-TEA-VV or GFP-VV. Tumor size was measured by caliper.

FIG. 12 shows that EphA2-TEA-VV resulted in a improved survival in A549 s.c. tumor model. The mice were inoculated with tumor followed by injection of VVs as described in FIG. 10. Mice survival was monitored (EphA2-TEA-VV n=8, GFP-VV n=7);

FIGS. 13A and 13B show that EphA2-TEA-VV results in an enhanced antitumor response in A549 i.v. lung cancer model. 2×10⁶ A549.eGFP.FFLuc cells were injected i.v. into SCID mice on day 0 and treated with a single injection of mixed 1×10⁷ unstimulated PBMC and 1×10⁸ PFU EphA2-TEA-VV or GFP-VV on day 7. Tumor progression was followed by in vivo bioluminescence imaging. A. Images of representative animals are shown. B. Solid lines represent each group of mice;

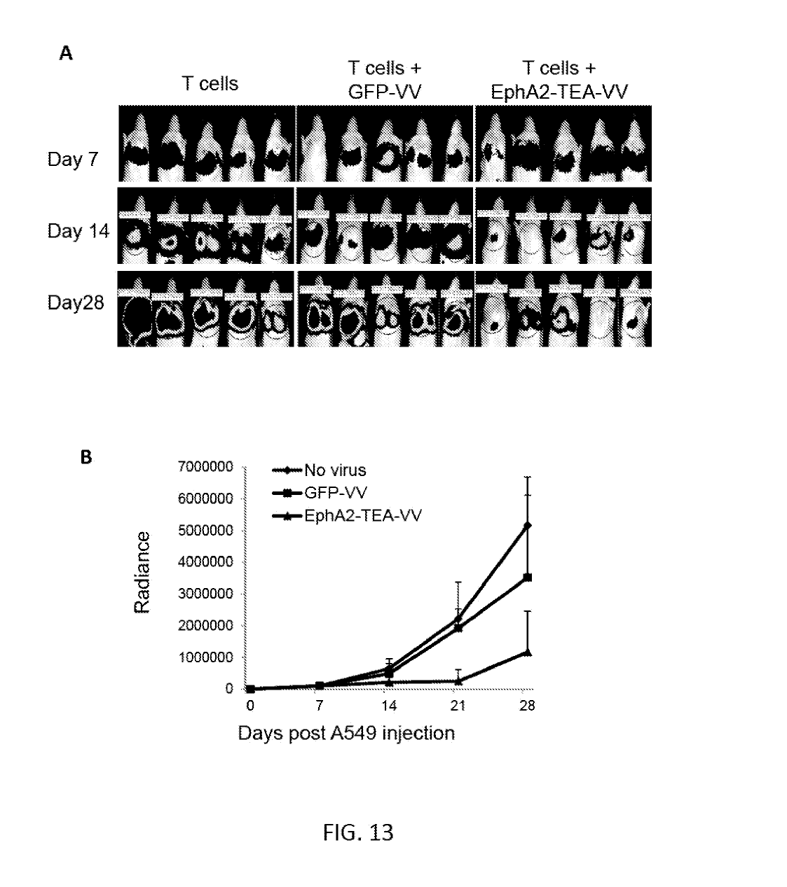

FIGS. 14A-14C demonstrate that HER2-TE induced killing of HER2+A549 tumor cells effectively. A. Scheme of lentiviral expression cassettes encoding HER2-scFv-CD3-scFv (HER2-TE) or GFP (GFP) in lentiviral vector. B. Mixed Lentivirus-infected A549 (GFP+, 0.6×10⁶/well) with non-infected A549 (GFP-, 0.4×10⁶/well) were co-cultured with unstimulated PBMC (1×10⁶/well) in 24-well plate for two days. The cells were harvested and stained with antibodies against CD3 followed by flow analysis. C. The percentage of cell populations was shown. D. GFP expression was examined under microscope;

FIGS. 15A-15B compare the ability of tandem bi-scFv and diabody of HER2-TE to induce killing of HER2+A549 (GFP+) tumor cells. A. Scheme of expression cassettes encoding tandem bi-scFv HER2-TE or diabody HER2-TE in lentiviral vector. B. Lentivirus-infected HER2+A549 tumor cells (1×10⁶/well) were co-cultured with unstimulated PBMC (1×10⁶/well) in 24-well plate. GFP expression was examined under microscope after 24 hours;

FIGS. 16A-16C show that GD2-TE induced killing of GD2+ JF and LAN1 tumor cells effectively. A. Scheme of expression cassettes encoding GD2-scFv-CD3-scFv (GD2-TE) or GFP (GFP) in lentiviral vector. Lentivirus-infected JF (GFP+) (B) or LAN1 (GFP+) (C) tumor cells (1×10⁶/well) were co-cultured with unstimulated PBMC (1×10⁶/well) in 24-well plate. GFP expression was examined under microscope.

FIG. 17 shows construction and function of HN3-TE and GC33-TE for Hepatocellular carcinoma (HCC). A. Scheme of expression cassettes encoding HN3-scFv-CD3-scFv (HN3-TE) or GC33-scFv-CD3-scFv (GC33-TE) in retroviral vector. B. HuH7 or HepG2 tumor cells (5×10³/well) were co-cultured with Retrovirus-infected human PBMC at different ratio of T cells to tumor cells, and tumor lysis was determined by a standard Cr release CTL assay.

FIG. 18 demonstrates how trimer or dimer form of 4-1BBL-armed T cell engager were constructed.

FIG. 19 illustrates enhanced antitumor effects of 4-1BBL-armed TEA-VV.

FIG. 20 demonstrates that co-stimulatory signaling 4-1BBL further enhanced tumor killing effects and induced T cell activation. Retrovirus (GFP, HER2-TE, dimer-41BBL-HER2-TE, or trimer-41BBL-TE)-infected A549 tumor cells (GFP+, 1×10⁶/well) were co-cultured with unstimulated PBMC, and 48 hours later, GFP expression was examined under microscope.

FIG. 21 shows cytokine ELISAs using IFNγ and IL2. Retrovirus (GFP, HER2-TE, dimer-41BBL-HER2-TE, or trimer-41BBL-TE)-infected A549 tumor cells (GFP+, 1×10⁶/well) were co-cultured with unstimulated PBMC, and 48 hours later, the culture medium were collected and cytokine expression of IFNγ and IL2 were examined by ELISA.

### DETAILED DESCRIPTION

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein.

### I. PARTICULAR EMBODIMENTS

The disclosure provides a novel oncolytic virus strategy, T-cell Engager-Armed Oncolytic Virus (TEA-OV), for the treatment of cancer, including advanced solid tumors. Oncolytic virus therapy has shown promise in preclinical models and in clinical studies as a novel cancer therapeutic. However, the antitumor efficacy is suboptimal and most tumors recurred, highlighting the need for further improvement. Oncolytic virus's major mode of action is the destruction of tumor cells, which may then induce antigen-specific T-cell responses against the tumor thereby targeting metastatic disease even after local injection of virus. At present, virus spread through the tumor and the induction of antigen-specific T-cell responses are limited, explaining the observed suboptimal antitumor activity of oncolytic virus. In embodiments of the disclosure, activating resident T cells within the tumor environment overcomes these limitations, because antigen-specific T cells can kill tumor cells that are not infected with vaccinia virus (by-stander killing), and the cytokines they release upon activation creates a pro-inflammatory microenvironment promoting the induction of endogenous tumor-specific T-cell responses.

To activate T cells within tumors, described herein are T-cell Engager Armed Oncolytic Viruses (TEA-OV) encoding a T-cell engager that is a bi-specific molecule comprising an activation domain and an antigen recognition domain. In specific cases, the engager comprises a CD3 single chain variable fragment (CD3-scFv). In an exemplary study using oncolytic vaccinia virus as a platform, CD3-scFvs were expressed as secretory bi-specific scFv that bind both to CD3 and an exemplary tumor cell surface antigen EphA2 (EphA2-TEA-VV). EphA2-TEA-VV displayed significantly enhanced tumor lytic activity by inducing bystander killing of tumor cells that are not infected with VVs, compared to control VVs. Thus, T-cell Engager Armed Oncolytic Vaccinia Virus (TEA-VV) encoding bi-specific scFvs bind both to CD3 and a tumor cell surface antigen displays significantly enhanced tumor lytic activity in the presence of T cells, representing a novel and enhanced oncolytic virus strategy with the unique ability of engaging T cells for cancer therapy.

Embodiments of the disclosure demonstrate that arming oncolytic VV with a T-cell engager (such as one that comprises at least CD3-scFv) results in enhanced antitumor effects, thereby opening a new avenue for development and use of effective oncolytic virus therapy. TEA-OV is useful for treatment of cancer, including advanced solid tumors that are often incurable with current treatment strategies.

### II. ENGAGER MOLECULES AND APPLICATIONS THEREOF

The oncolytic viral vectors provided herein are, in certain embodiments, genetically engineered to include a polynucleotide sequence that encodes an engager molecule, e.g., an engager polypeptide. Such engager polypeptides generally comprise an antigen recognition domain and an activation domain. The engager molecule's antigen recognition domain may be designed so as to bind to one or more molecules present on target cells, while engager molecule's activation domain binds to a molecule present on effector cells, such as T lymphocytes, for example. Once the engager molecule's activation domain has bound effector cells, the activation domain can activate the effector cells. In certain embodiments, when the activation domain of the engager binds to the activation molecule on the immune cell, and the antigen recognition domain binds to the target-cell antigen, the immune cell kills the target cell.

The engager may be bipartite (e.g., comprising an activation domain and antigen recognition domain that may optionally be joined by a linker), or may be tripartite or multipartite (e.g., comprise one or more activation domains and/or antigen recognition domains, or other domains, including one or more co-stimulatory domains and/or one or more dimerization or trimerization domains).

In certain embodiments, the engager is a protein, e.g., an engineered protein. In specific embodiments, the activation domain of the engager is or comprises an antibody or an antigen-binding fragment or portion thereof, e.g., a single chain variable fragment (scFv). On other specific embodiments, the antigen recognition domain is or comprises an antibody or an antibody fragment or an antigen-binding fragment or portion thereof, e.g., a monoclonal antibody, Fv, or an scFv, or it may comprise ligands, peptides, soluble T-cell receptors, or combinations thereof. In certain embodiments, the activation domain and antigen recognition domain are joined by a linker, e.g., a peptide linker.

The activation domain of an engager molecule can provide activation to immune cells. The skilled artisan recognizes that immune cells have different activating receptors. For example CD3 is an activating receptor on T-cells, whereas CD16, NKG2D, or NKp30 are activating receptors on NK cells, and CD3 or an invariant TCR are the activating receptors on NKT-cells. Engager molecules that activate T-cells may therefore have a different activation domain than engager molecules that activate NK cells. In specific embodiments, e.g., wherein the immune cell is a T-cell, the activation molecule is one or more of CD3, e.g., CD3γ, CD3δ or CD3ε; or CD27, CD28, CD40, CD134, CD137, and CD278. In other specific embodiments, e.g., wherein the immune cell is a NK cell, the activation molecule is CD16, NKG2D, or NKp30, or wherein the immune cell is a NKT-cell, the activation molecule is CD3 or an invariant TCR.

In certain other embodiments, the engager additionally comprises one or more other domains, e.g., one or more of a cytokine, a costimulatory domain, a domain that inhibits negative regulatory molecules of T-cell activation, or a combination thereof. In specific embodiments, the cytokine is IL-15, IL-2, and/or IL-7. In other specific embodiments, the co-stimulatory domain is CD27, CD80, CD83, CD86, CD134, or CD137. In other specific embodiments, the domain that inhibits negative regulatory molecules of T-cell activation is PD-1, PD-L1, CTLA4, or B7-H4.

Examples of Specific Engager Molecules. Below are provided specific examples of engager molecules. In general, an scFv contains a VH and VL domain connected by a linker peptide. For example, SEQ ID NO:1 is a EphA2-CD3 T-cell Engager comprising the following formula:

EphA2-CD3 T-Cell Engager

Leader-VH4H5-(G4S1)3-VL4H5-SG4S-VHOKT3-(G4S1)3-VLOKT3

SEQ ID NO:1 is as follows, where the first underlined section is the leader and the following underlined sections are linker sequences between the respective scFvs according to the formula above:

MDWIWRILFLVGAATGAHSQVQLLESGGGLVQPGGSLRLSCAASGFTFSS
YTMSWVRQAPGQALEWMGTISSGGTYTYYPDSVKGRFTISRDNAKNSLYL
QMNSLRAEDTAVYYCAREAIFTYWGRGTLVTSSGGGGSGGGGSGGGGSDI
QLTQSPSSLSASVGDRVTITCKASQDINNYLSWYQQKPGQAPRLLIYRAN
RLVDGVPDRFSGSGYGTDFTLTINNIESEDAAYYFCLKYDVFPYTFGQGT
KVEIKSGGGGSDIKLQQSGAELARPGASVKMSCKTSGYTFTRYTMHWVKQ
RPGQGLEWIGYINPSRGYTNYNQKFKDKATLTTDKSSSTAYMQLSSLTSE
DSAVYYCARYYDDHYCLDYWGQGTTLTVSSGGGGSGGGGSGGGGSDIQLT
QSPAIMSASPGEKVTMTCRASSSVSYMNWYQQKSGTSPKRWIYDTSKVAS
GVPYRFSGSGSGTSYSLTISSMEAEDAATYYCQQWSSNPLTFGAGTKLEL
KS

HER2-CD3 T-Cell Engager

Leader-VHFRP5-(G4S1)3-VLFRP5-G4S-VHOKT3-(G4S1)3-VLOKT3

SEQ ID NO:2 is as follows, where the first underlined section is the leader and the following underlined sections are linker sequences between the respective scFvs according to the formula above:

MDWIWRILFLVGAATGAHSEVQLQQSGPELKKPGETVKISCKASGYPFTN
YGMNWVKQAPGQGLKWMGWINTSTGESTFADDFKGRFDFSLETSANTAYL
QINNLKSEDMATYFCARWEVYHGYVPYWGQGTTVTVSSGGGGSGGGGSGG
GGSDIQLTQSHKFLSTSVGDRVSITCKASQDVYNAVAWYQQKPGQSPKLL
IYSASSRYTGVPSRFTGSGSGPDFTFTISSVQAEDLAVYFCQQHFRTPFT
FGSGTKLEIKALGGGGSDIKLQQSGAELARPGASVKMSCKTSGYTFTRYT
MHWVKQRPGQGLEWIGYINPSRGYTNYNQKFKDKATLTTDKSSSTAYMQL
SSLTSEDSAVYYCARYYDDHYCLDYWGQGTTLTVSSGGGGSGGGGSGGGG
SDIQLTQSPAIMSASPGEKVTMTCRASSSVSYMNWYQQKSGTSPKRWIYD
TSKVASGVPYRFSGSGSGTSYSLTISSMEAEDAATYYCQQWSSNPLTFGA
GTKLELKS

GD2-CD3 T-Cell Engager

Leader-VH14g2a-(SG4SG2)-VL14g2a-G4S-VHOKT3-(G4S1)3-VLOKT3

SEQ ID NO:3 is as follows, where the first underlined section is the leader and the following underlined sections are linker sequences between the respective scFvs according to the formula above:

MEFGLSWLFLVAILKGVQCSRDILLTQTPLSLPVSLGDQASISCRSSQSL
VHRNGNTYLHWYLQKPGQSPKLLIHKVSNRFSGVPDRFSGSGSGTDFTLK
ISRVEAEDLGVYFCSQSTHVPPLTFGAGTKLELKRADAAPTVSIFPGSGG
GGSGGEVKLQQSGPSLVEPGASVMISCKASGSSFTGYNMNWVRQNIGKSL
EWIGAIDPYYGGTSYNQKFKGRATLTVDKSSSTAYMHLKSLTSEDSAVYY
CVSGMEYWGQGTSVTVSSGGGGSDIKLQQSGAELARPGASVKMSCKTSGY
TFTRYTMHWVKQRPGQGLEWIGYINPSRGYTNYNQKFKDKATLTTDKSSS
TAYMQLSSLTSEDSAVYYCARYYDDHYCLDYWGQGTTLTVSSGGGGSGGG
GSGGGGSDIQLTQSPAIMSASPGEKVTMTCRASSSVSYMNWYQQKSGTSP
KRWIYDTSKVASGVPYRFSGSGSGTSYSLTISSMEAEDAATYYCQQWSSN
PLTFGAGTKLELKS

HN3-CD3 T-Cell Engager

Leader-VHHN3-G4S-VHOKT3-(G4S1)3-VLOKT3

SEQ ID NO:4 is as follows, where the first underlined section is the leader and the following underlined sections are linker sequences between a VH and a scFv according to the formula above:

MDWIWRILFLVGAATGAHQVQLVQSGGGLVQPGGSLRLSCAASYFDFDSY
EMSWVRQAPGKGLEWIGSIYHSGSTYYNPSLKSRVTISRDNSKNTLYLQM
NTLRAEDTATYYCARVNMDRFDYWGQGTLVTVSSSGGGGSDIKLQQSGAE
LARPGASVKMSCKTSGYTFTRYTMHWVKQRPGQGLEWIGYINPSRGYTNY
NQKFKDKATLTTDKSSSTAYMQLSSLTSEDSAVYYCARYYDDHYCLDYWG
QGTTLTVSSGGGGSGGGGSGGGGSDIQLTQSPAIMSASPGEKVTMTCRAS
SSVSYMNWYQQKSGTSPKRWIYDTSKVASGVPYRFSGSGSGTSYSLTISS
MEAEDAATYYCQQWSSNPLTFGAGTKLELKS

GC33-CD3 T-cell Engager

Leader-VHGC33-(G4S1)3-VLGC33-G4S-VHOKT3-(G4S1)3-VLOKT3

SEQ ID NO:5 is as follows, where the first underlined section is the leader and the following underlined sections are linker sequences between the respective scFvs according to the formula above:

MDWIWRILFLVGAATGAHSQVQLQQSGAELVRPGASVKLSCKASGYTFTD
YEMHWVKQTPVHGLKWIGALDPKTGDTAYSQKFKGKATLTADKSSSTAYM
ELRSLTSEDSAVYYCTRFYSYTYWGQGTLVTVSAGGGGSGGGGSGGGGSD
VVMTQTPLSLPVSLGDQASISCRSSQSLVHSNGNTYLHWYLQKPGQSPKL
LIYKVSNRFSGVPDRFSGSGSGTDFTLKISRVEAEDLGVYFCSQNTHVPP
TFGSGTKLEIKSGGGGSDIKLQQSGAELARPGASVKMSCKTSGYTFTRYT
MHWVKQRPGQGLEWIGYINPSRGYTNYNQKFKDKATLTTDKSSSTAYMQL
SSLTSEDSAVYYCARYYDDHYCLDYWGQGTTLTVSSGGGGSGGGGSGGGG
SDIQLTQSPAIMSASPGEKVTMTCRASSSVSYMNWYQQKSGTSPKRWIYD
TSKVASGVPYRFSGSGSGTSYSLTISSMEAEDAATYYCQQWSSNPLTFGA
GTKLELKS

Dimerized 41BBL-HER2-CD3 T-Cell Engager

Leader-VHGC33-(G4S1)3-VLGC33-CH2CH3-(G4S1)-41BBL-(G4S1)-VHOKT3-(G4S1)3-VLOKT3

SEQ ID NO:6 is as follows, where the first underlined section is the leader and the following underlined sections are linker sequences between the respective scFvs or CH2CH3 domain or 4-1BBL according to the formula above:

MDWIWRILFLVGAATGAHSEVQLQQSGPELKKPGETVKISCKASGYPFTN
YGMNWVKQAPGQGLKWMGWINTSTGESTFADDFKGRFDFSLETSANTAYL
QINNLKSEDMATYFCARWEVYHGYVPYWGQGTTVTVSSGGGGSGGGGSGG
GGSDIQLTQSHKFLSTSVGDRVSITCKASQDVYNAVAWYQQKPGQSPKLL
IYSASSRYTGVPSRFTGSGSGPDFTFTISSVQAEDLAVYFCQQHFRTPFT
FGSGTKLEIKALFEEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGKGGGG
SASACPWAVSGARASPGSAASPRLREGPELSPDDPAGLLDLRQGMFAQLV
AQNVLLIDGPLSWYSDPGLAGVSLTGGLSYKEDTKELVVAKAGVYYVFFQ
LELRRVVAGEGSGSVSLALHLQPLRSAAGAAALALTVDLPPASSEARNSA
FGFQGRLLHLSAGQRLGVHLHTEARARHAWQLTQGATVLGLFRVTPEIPA
GLPSPRSEGSGGGGSGGGGSGGGGSVDDIKLQQSGAELARPGASVKMSCK
TSGYTFTRYTMHWVKQRPGQGLEWIGYINPSRGYTNYNQKFKDKATLTTD
KSSSTAYMQLSSLTSEDSAVYYCARYYDDHYCLDYWGQGTTLTVSSGGGG
SGGGGSGGGGSDIQLTQSPAIMSASPGEKVTMTCRASSSVSYMNWYQQKS
GTSPKRWIYDTSKVASGVPYRFSGSGSGTSYSLTISSMEAEDAATYYCQQ
WSSNPLTFGAGTKLELKS

Trimerized 41BBL-HER2-CD3 T-Cell Engager

Leader-VHGC33-(G4S1)3-VLGC33-LINKER-Col-LINKER-41BBL-(G4S1)-VHOKT3-(G4S1)3-VLOKT3

SEQ ID NO:7 is as follows, where the first underlined section is the leader and the following underlined sections are linker sequences between the respective scFvs or Col domain or 4-1BBL according to the formula above:

MDWIWRILFLVGAATGAHSEVQLQQSGPELKKPGETVKISCKASGYPFTN
YGMNWVKQAPGQGLKWMGWINTSTGESTFADDFKGRFDFSLETSANTAYL
QINNLKSEDMATYFCARWEVYHGYVPYWGQGTTVTVSSGGGGSGGGGSGG
GGSDIQLTQSHKFLSTSVGDRVSITCKASQDVYNAVAWYQQKPGQSPKLL
IYSASSRYTGVPSRFTGSGSGPDFTFTISSVQAEDLAVYFCQQHFRTPFT
FGSGTKLEIKALFEGAGGSGGSSGSDGASGSRVTAFSNMDDMLQKAHLVI
EGTFIYLRDSTEFFIRVRDGWKKLQLGELIPIPADSPPPPALSSNPGAGG
SGGSSGSDGASGSRASACPWAVSGARASPGSAASPRLREGPELSPDDPAG
LLDLRQGMFAQLVAQNVLLIDGPLSWYSDPGLAGVSLTGGLSYKEDTKEL
VVAKAGVYYVFFQLELRRVVAGEGSGSVSLALHLQPLRSAAGAAALALTV
DLPPASSEARNSAFGFQGRLLHLSAGQRLGVHLHTEARARHAWQLTQGAT
VLGLFRVTPEIPAGLPSPRSEGSGGGGSGGGGSGGGGSVDDIKLQQSGAE
LARPGASVKMSCKTSGYTFTRYTMHWVKQRPGQGLEWIGYINPSRGYTNY
NQKFKDKATLTTDKSSSTAYMQLSSLTSEDSAVYYCARYYDDHYCLDYWG
QGTTLTVSSGGGGSGGGGSGGGGSDIQLTQSPAIMSASPGEKVTMTCRAS
SSVSYMNWYQQKSGTSPKRWIYDTSKVASGVPYRFSGSGSGTSYSLTISS
MEAEDAATYYCQQWSSNPLTFGAGTKLELKS

For any of the engagers described herein, the respective domains may be in any order N-terminus to C-terminus, including, e.g., having the activation domain N-terminal to the antigen recognition domain, having the activation domain C-terminal to the antigen recognition domain, and so forth. In certain embodiments, T-cells are modified to secrete engager molecules that have the antigen recognition domain N-terminal to the activation domain. In particular embodiments, two or more of the domains of an engager molecule are separated by a linker. The linker may be of any suitable length, and such a parameter is routinely optimized in the art. For example, linkers may be of a length and sequence sufficient to ensure that each of the first and second domains can, independently from one another, retain their differential binding specificities.

In certain embodiments, the antigen bound by the antigen recognition domain is a tumor-associated antigen (TAA) or a tumor-specific antigen (TSA). In certain embodiments, the antigen recognition domain of the engager is an scFv that is specific for a TAA or TSA. In a specific embodiment, the TAA or TSA is expressed on a cancer cell. In one embodiment, the TAA or TSA is expressed on a blood cancer cell. In another embodiment, the TAA or TSA is expressed on a cell of a solid tumor. In more specific embodiments, the solid tumor is a glioblastoma, a non-small cell lung cancer, a lung cancer other than a non-small cell lung cancer, breast cancer, prostate cancer, pancreatic cancer, liver cancer, colon cancer, stomach cancer, a cancer of the spleen, skin cancer, a brain cancer other than a glioblastoma, a kidney cancer, a thyroid cancer, or the like. In more specific embodiments, the TAA or TSA is expressed by a tumor cell in an individual. In specific embodiments, the TAA or TSA is one or more of, e.g., an scFv on the engager is specific for one or more of EphA2, HER2, GD2, Glypican-3, 5T4, 8H9, αᵥβ₆ integrin, B7-H3, B7-H6, CAIX, CA9, CD19, CD20, CD22, kappa light chain, CD30, CD33, CD38, CD44, CD44v6, CD44v7/8, CD70, CD123, CD138, CD171, CEA, CSPG4, EGFR, EGFRvIII, EGP2, EGP40, EPCAM, ERBB3, ERBB4, ErbB3/4, FAP, FAR, FBP, fetal AchR, Folate Receptor a, GD2, GD3, HLA-AI MAGE A1, HLA-A2, IL11Ra, IL13Ra2, KDR, Lambda, Lewis-Y, MCSP, Mesothelin, Muc1, Muc16, NCAM, NKG2D ligands, NY-ESO-1, PRAME, PSCA, PSC1, PSMA, ROR1, SURVIVIN, TAG72, TEM1, TEM8, VEGRR2, carcinoembryonic antigen, HMW-MAA, VEGF receptors, and other exemplary antigens are antigens that are present with in the extracellular matrix of tumors, such as oncofetal variants of fibronectin, tenascin, or necrotic regions of tumors.

In embodiments of the disclosure, methods and compositions relate to compositions comprising a bi-specific single chain antibody construct. The bi-specific single chain antibody construct may comprise or consist of or consist essentially of at least two domains, whereby one of said at least two domains specifically binds to a tumor antigen such as EphA2, HER2, GD2 or Glypican-3, for example, and a second domain binds to a T cell protein, such as CD3 antigen, for example.

EphA2 may be referred to as EPH receptor A2 (ephrin type-A receptor 2; EPHA2; ARCC2; CTPA; CTPP1; or ECK), which is a protein that in humans is encoded by the EPHA2 gene in the ephrin receptor subfamily of the protein-tyrosine kinase family. Receptors in this subfamily generally comprise a single kinase domain and an extracellular region comprising a Cys-rich domain and 2 fibronectin type III repeats; embodiments of the antibodies of the disclosure may target any of these domains. The ephrin receptors are divided into two groups as a result of the similarity of their respective extracellular domain sequences and also their affinities for binding ephrin-A and ephrin-B ligands, and EphA2 encodes a protein that binds ephrin-A ligands. An exemplary human EphA2 nucleic sequence is in GenBank® Accession No. NM_{—}004431, and an exemplary human EphA2 polypeptide sequence is in GenBank® Accession No. NP_{—}004422, both of which sequences are incorporated herein in their entirety.

HER2 may be referred to as human Epidermal Growth Factor Receptor 2 (Neu, ErbB-2, CD340, or p185), which is a protein that in humans is encoded by the ERBB2 gene in the epidermal growth factor receptor (EFR/ErbB) family. HER2 contains an extracellular ligand binding domain, a transmembrane domain, and an intracellular domain that can interact with a multitude of signaling molecules. Embodiments of the antibodies of the disclosure may target the extracellular ligand binding domain.

An exemplary human EphA2 nucleic sequence is in GenBank® Accession No. NM_{—}004448.2, and an exemplary human EphA2 polypeptide sequence is in GenBank® Accession No. NP_{—}004439, both of which sequences are incorporated herein in their entirety.

GD2 is a disialoganglioside expressed on tumors of neuroectodermal origin, including human neuroblastoma and melanoma, with highly restricted expression on normal tissues, principally to the cerebellum and peripheral nerves in humans. GD2 is present and concentrated on cell surfaces, with the two hydrocarbon chains of the ceramide moiety embedded in the plasma membrane and the oligosaccharides located on the extracellular surface, where they present points of recognition for extracellular molecules or surfaces of neighboring cells. Embodiments of the antibodies of the disclosure may target the extracellular domain.

The term “bispecific single chain antibody construct” relates to a construct comprising two antibody derived binding domains. In specific embodiments, bi-specific single chain antibody construct may be tandem bi-scFv or diabody. One of the binding domains may comprise variable regions (or parts thereof) of both heavy chain (VH) and light chain (VL) of an antibody, antibody fragment or derivative thereof, capable of specifically binding to/interacting with EphA2, HER2, GD2 or Glypican-3, for example. The second binding domain may comprise variable regions (or parts thereof) of both heavy chain (VH) and light chain (VL) of an antibody, antibody fragment or derivative thereof, capable of specifically binding to/interacting with human CD3 antigen, for example. In specific embodiments, part of a variable region may be at least one CDR (“Complementary determining region”), such as at least the CDR3 region.

In specific embodiments, the “bispecific single chain antibody construct” to be employed in the composition of the disclosure is a bispecific single chain Fv (scFv). A scFv in general contains a VH and VL domain connected by a linker peptide. The secretable Engager is composed of a Signal peptide (to allow for secretion) from cells, followed by 2 scFvs connected by linker peptides (Lx, Ly, Lz). Linkers may be of a length and sequence sufficient to ensure that each of the first and second domains can, independently from one another, retain their differential binding specificities. Bispecific single chain molecules are known in the art and are described in WO 99/54440; Mack, J. Immunol. (1997), 158, 3965-3970; Mack, PNAS, (1995), 92, 7021-7025; Kufer, Cancer Immunol. Immunother., (1997), 45, 193-197; Loffler, Blood, (2000), 95, 6, 2098-2103; and Bruhl, J. Immunol., (2001), 166, 2420-2426.

In specific embodiments of the disclosure, an exemplary molecular format of the disclosure provides an oncolytic viral polynucleotide construct encoding a polypeptide that comprises a signal peptide followed by two antibody-derived regions. Each antibody-derived region (scFv) comprises one V_{H} and one V_{L} region. In specific embodiments, bi-specific scFv may be tandem bi-scFv or diabody. Bispecific ScFvs can be arranged in different formats: V_{H}α-Lx-V_{L}α-Ly-V_{H}β-Lz-V_{L}β, V_{L}α-Lx-V_{H}α-Ly-V_{H}β-Lz-V_{L}β, V_{L}α-Lx-V_{H}α-Ly-V_{L}β-Lz-V_{H}β, V_{H}α-Lx-V_{L}α-Ly-V_{L}β-Lz-V_{H}β, V_{H}α-Lx-V_{L}β-Ly-V_{H}β-Lz-V_{L}α, V_{L}α-Lx-V_{L}β-Ly-V_{H}β-Lz-V_{H}α, V_{H}α-Lx-V_{H}β-Ly-V_{L}β-Lz-V_{L}α, V_{L}α-Lx-V_{H}β-Ly-V_{L}β-Lz-V_{H}α, V_{H}β-Lx-V_{L}α-Ly-V_{H}α-Lz-V_{L}β, V_{L}β-Lx-V_{L}α-Ly-V_{H}α-Lz-V_{H}β, V_{H}β-Lx-V_{H}α-Ly-V_{L}α-Lz-V_{L}β, V_{L}β-Lx-V_{H}α-Ly-V_{L}α-Lz-V_{H}β. Thus, bispecific scFvs with the above possible arrangements are particular embodiments of the bispecific single chain construct.

In specific embodiments of the disclosure, the antibody construct may also comprise additional domains, e.g. for the isolation and/or preparation of recombinantly produced constructs. In addition, the antigen binding domain may contain multiple antigen recognition binding domains allowing targeting of multiple antigens, while the activation domain may contain multiple domains to activate cells.

The term “single-chain” as used in accordance with the present disclosure means that said first and second domain of the bi-specific single chain construct are covalently linked, preferably in the form of a co-linear amino acid sequence encodable by a single nucleic acid molecule.

The term “binding to/interacting with” as used in the context with the present disclosure defines a binding/interaction of at least two “antigen-interaction-sites” with each other. The term “antigen-interaction-site” defines, in accordance with the present disclosure, a motif of a polypeptide that shows the capacity of specific interaction with a specific antigen or a specific group of antigens. The binding/interaction is also understood to define a “specific recognition”. The term “specifically recognizing” means in accordance with this disclosure that the antibody molecule is capable of specifically interacting with and/or binding to at least two amino acids of each of the target molecules as defined herein. The term relates to the specificity of the antibody molecule, i.e. to its ability to discriminate between the specific regions of the human target molecule as defined herein. The specific interaction of the antigen-interaction-site with its specific antigen may result in an initiation of a signal, e.g. due to the induction of a change of the conformation of the antigen, an oligomerization of the antigen, etc. Further, the binding may be exemplified by the specificity of a “key-lock-principle”. Thus, specific motifs in the amino acid sequence of the antigen-interaction-site and the antigen bind to each other as a result of their primary, secondary or tertiary structure as well as the result of secondary modifications of said structure, in some embodiments. The specific interaction of the antigen-interaction-site with its specific antigen may result as well in a simple binding of the site to the antigen.

The term “specific interaction” as used in accordance with the present disclosure means that the bi-specific single chain construct does not or essentially does not cross-react with (poly)peptides of similar structures. Cross-reactivity of a panel of bi-specific single chain construct under investigation may be tested, for example, by assessing binding of the panel of bi-specific single chain construct under conventional conditions (see, e.g., Harlow and Lane, Antibodies: A Laboratory Manual, Cold Spring Harbor Laboratory Press, 1988 and Using Antibodies: A Laboratory Manual, Cold Spring Harbor Laboratory Press, 1999) to the (poly)peptide of interest as well as to a number of more or less (structurally and/or functionally) closely related (poly)peptides. Only those antibodies that bind to the (poly)peptide/protein of interest but do not or do not essentially bind to any of the other (poly)peptides are considered specific for the (poly)peptide/protein of interest. Examples for the specific interaction of an antigen-interaction-site with a specific antigen comprise the specificity of a ligand for its receptor. The definition particularly comprises the interaction of ligands which induce a signal upon binding to its specific receptor. Examples for corresponding ligands comprise cytokines that interact/bind with/to its specific cytokine-receptors. Another example for said interaction, which is also particularly comprised by said definition, is the interaction of an antigenic determinant (epitope) with the antigenic binding site of an antibody.

The term “binding to/interacting with” may also relate to a conformational epitope, a structural epitope or a discontinuous epitope consisting of two regions of the human target molecules or parts thereof. In context of this disclosure, a conformational epitope is defined by two or more discrete amino acid sequences separated in the primary sequence which come together on the surface of the molecule when the polypeptide folds to the native protein (Sela, (1969) Science 166, 1365 and Layer, (1990) Cell 61, 553-6).

The constructs of the present disclosure are also envisaged to specifically bind to/interact with a conformational/structural epitope(s) composed of and/or comprising the two regions of the human CD3 complex described herein or parts thereof as disclosed herein below.

Accordingly, specificity can be determined experimentally by methods known in the art and methods as disclosed and described herein. Such methods comprise, but are not limited to Western blots, ELISA-, RIA-, ECL-, IRMA-, EIA-tests and peptide scans.

The term “antibody fragment or derivative thereof” relates to single chain antibodies, or fragments thereof, synthetic antibodies, antibody fragments, such as a Camel Ig, Ig NAR, Fab fragments, Fab′ fragments, F(ab)′2 fragments, F(ab)′3 fragments, Fv, single chain Fv antibody (“scFv”), bis-scFv, (scFv)2, minibody, diabody, triabody, tetrabody, disulfide stabilized Fv protein (“dsFv”), and single-domain antibody (sdAb, nanobody), etc., or a chemically modified derivative of any of these. Antibodies to be employed in accordance with the disclosure or their corresponding immunoglobulin chain(s) can be further modified using conventional techniques known in the art, for example, by using amino acid deletion(s), insertion(s), substitution(s), addition(s), and/or recombination(s) and/or any other modification(s) (e.g. posttranslational and chemical modifications, such as glycosylation and phosphorylation) known in the art either alone or in combination. Methods for introducing such modifications in the DNA sequence underlying the amino acid sequence of an immunoglobulin chain are well known to the person skilled in the art; see, e.g., Sambrook et al. (1989).

The term “antibody fragment or derivative thereof” particularly relates to (poly)peptide constructs comprising at least one CDR.

Fragments or derivatives of the recited antibody molecules define (poly)peptides that are parts of the above antibody molecules and/or which are modified by chemical/biochemical or molecular biological methods. Corresponding methods are known in the art and described inter alia in laboratory manuals (see Sambrook et al.; Molecular Cloning: A Laboratory Manual; Cold Spring Harbor Laboratory Press, 2nd edition 1989 and 3rd edition 2001; Gerhardt et al.; Methods for General and Molecular Bacteriology; ASM Press, 1994; Lefkovits; Immunology Methods Manual: The Comprehensive Sourcebook of Techniques; Academic Press, 1997; Golemis; Protein-Protein Interactions: A Molecular Cloning Manual; Cold Spring Harbor Laboratory Press, 2002).

Variable domains comprised in the herein described bi-specific single chain constructs may be connected by additional linker sequences. The term “peptide linker” defines in accordance with the present disclosure an amino acid sequence by which the amino acid sequences of the first domain and the second domain of the defined construct are linked with each other. An essential technical feature of such peptide linker is that said peptide linker does not comprise any polymerization activity. The characteristics of a peptide linker, which comprise the absence of the promotion of secondary structures, are known in the art and described, e.g., in Dall'Acqua et al. (Biochem. (1998) 37, 9266-9273), Cheadle et al. (Mol Immunol (1992) 29, 21-30) and Raag and Whitlow (FASEB (1995) 9(1), 73-80). An envisaged peptide linker with less than 5 amino acids can comprise 4, 3, 2 or one amino acids. A particularly preferred “single” amino acid in context of the “peptide linker” is Gly. Accordingly, the peptide linker may consist of the single amino acid Gly. Furthermore, peptide linkers that also do not promote any secondary structures are preferred. The linkage of the domains to each other can be provided by, e.g., genetic engineering. Methods for preparing fused and operatively linked bispecific single chain constructs and expressing them in mammalian cells or bacteria are well-known in the art (e.g. WO 99/54440, Ausubel, Current Protocols in Molecular Biology, Green Publishing Associates and Wiley Interscience, N. Y. 1989 and 1994 or Sambrook et al., Molecular Cloning: A Laboratory Manual, Cold Spring Harbor Laboratory Press, Cold Spring Harbor, N. Y., 2001).

The bi-specific single chain antibody constructs described herein above and below may be humanized or deimmunized antibody constructs. Methods for the humanization and/or deimmunization of (poly))peptides and, in particular, antibody constructs are known to the person skilled in the art.

In one embodiment of the pharmaceutical composition of this disclosure, the V_{H} and V_{L} regions of the human CD3 specific domain are derived from an CD3 specific antibody selected from the group consisting of X35-3, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, WT31 and F101.01. These CD3-specific antibodies are well known in the art and, inter alia, described in Tunnacliffe (1989), Int. Immunol. 1, 546-550. In specific embodiments, V_{H} and V_{L} regions are derived from antibodies/antibody derivatives and the like that are capable of specifically recognizing the human CD3-ε chain or human CD3-ζ chain.

The present disclosure also encompasses a composition comprising an oncolytic viral nucleic acid sequence encoding a bi-specific single chain antibody construct as defined above and cells harboring the nucleic acid sequence. The oncolytic viral nucleic acid molecule is a recombinant nucleic acid molecule, in particular aspects and may be synthetic.

In another aspect, provided herein is a virus polynucleotide that encodes an engager molecule, e.g., any of the engager molecules described herein. In a specific embodiment, the viral polynucleotide allows inducible expression of the engager molecule in a cell that contains the virus. In any of the embodiments provided herein, the virus preferably encodes the engager in a form that is secretable by the cell. The polynucleotide encodes a fusion molecule comprising an activation domain and an antigen recognition domain, and in specific embodiments each domain is a scFv. The activation domain may be positioned toward the N-terminus of the polypeptide in relation to the antigen recognition domain, or the activation domain may be positioned toward the C-terminus of the polypeptide in relation to the antigen recognition domain.

It is evident to the person skilled in the art that regulatory sequences may be added to the oncolytic viral nucleic acid molecule comprised in the composition of the disclosure. For example, promoters, transcriptional enhancers and/or sequences that allow for induced expression of the polynucleotide of the disclosure may be employed. A suitable inducible system is for example tetracycline-regulated gene expression as described, e.g., by Gossen and Bujard (Proc. Natl. Acad. Sci. USA 89 (1992), 5547-5551) and Gossen et al. (Trends Biotech. 12 (1994), 58-62), or a dexamethasone-inducible gene expression system as described, e.g. by Crook (1989) EMBO J. 8, 513-519.

Furthermore, it is envisaged for further purposes that oncolytic viral nucleic acid molecules may contain, for example, thioester bonds and/or nucleotide analogues. The modifications may be useful for the stabilization of the nucleic acid molecule against endo- and/or exonucleases in the cell. The nucleic acid molecules may be transcribed by an appropriate oncolytic vector comprising a chimeric gene that allows for the transcription of the nucleic acid molecule in the cell. In this respect, it is also to be understood that such polynucleotides can be used for “gene targeting” or “gene therapeutic” approaches. In another embodiment the nucleic acid molecules are labeled. Methods for the detection of nucleic acids are well known in the art, e.g., Southern and Northern blotting, PCR or primer extension. This embodiment may be useful for screening methods for verifying successful introduction of the nucleic acid molecules described above, for example during gene therapy approaches.

The oncolytic viral nucleic acid molecule(s) may be a recombinantly produced chimeric nucleic acid molecule comprising any of the aforementioned nucleic acid molecules either alone or in combination. In specific aspects, the nucleic acid molecule is part of a vector.

The present disclosure therefore also relates to a composition comprising an oncolytic viral vector comprising the nucleic acid molecule described in the present disclosure.

In specific embodiments, there is an oncolytic viral vector that comprises a nucleic acid sequence that is a regulatory sequence operably linked to the nucleic acid sequence encoding a bi-specific single chain antibody constructs defined herein. Such regulatory sequences (control elements) are known to the artisan and may include a promoter, a splice cassette, translation initiation codon, translation and insertion site for introducing an insert into the vector. In specific embodiments, the nucleic acid molecule is operatively linked to said expression control sequences allowing expression in eukaryotic or prokaryotic cells.

It is envisaged that an oncolytic viral vector is an expression vector comprising the nucleic acid molecule encoding a bi-specific single chain antibody constructs defined herein. In specific aspects, the oncolytic viral vector is a vaccinia viral vector. The vaccinia virus may be the Wyeth or Western Reserve (WR) strain. The vaccinia virus may have a deletion in its genome or a mutation in one or more genes. The thymidine kinase gene of the vaccinia virus may have been deleted. The vaccinia virus may have a mutation in a gene encoding vaccinia virus growth factor. In specific aspects, the oncolytic viral vector is a lentiviral vector. Lentiviral vectors are commercially available, including from Clontech (Mountain View, Calif.) or GeneCopoeia (Rockville, Md.), for example.

The term “regulatory sequence” refers to DNA sequences that are necessary to effect the expression of coding sequences to which they are ligated. The nature of such control sequences differs depending upon the host organism. In prokaryotes, control sequences generally include promoters, ribosomal binding sites, and terminators. In eukaryotes generally control sequences include promoters, terminators and, in some instances, enhancers, transactivators or transcription factors. The term “control sequence” is intended to include, at a minimum, all components the presence of which are necessary for expression, and may also include additional advantageous components.

The term “operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences. In case the control sequence is a promoter, it is obvious for a skilled person that double-stranded nucleic acid is preferably used.

Thus, the recited vector is an oncolytic viral expression vector, in certain embodiments. An “expression vector” is a construct that can be used to transform a selected host and provides for expression of a coding sequence in the selected host. Expression vectors can for instance be cloning vectors, binary vectors or integrating vectors. Expression comprises transcription of the nucleic acid molecule preferably into a translatable mRNA. Regulatory elements ensuring expression in eukaryotic cells are well known to those skilled in the art. In the case of eukaryotic cells they comprise normally promoters ensuring initiation of transcription and optionally poly-A signals ensuring termination of transcription and stabilization of the transcript. Examples of regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells.

Beside elements that are responsible for the initiation of transcription such regulatory elements may also comprise transcription termination signals, such as the SV40-poly-A site or the tk-poly-A site, downstream of the polynucleotide. Furthermore, depending on the expression system used leader sequences capable of directing the polypeptide to a cellular compartment or secreting it into the medium may be added to the coding sequence of the recited nucleic acid sequence and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a fusion protein including an N-terminal identification peptide imparting desired characteristics, e.g., stabilization or simplified purification of expressed recombinant product; see supra. In this context, suitable expression vectors are known in the art such as Okayama-Berg cDNA expression vector pcDV1 (Pharmacia), pEF-Neo, pCDM8, pRc/CMV, pcDNA1, pcDNA3 (Invitrogen), pEF-DHFR and pEF-ADA, (Raum et al. Cancer Immunol Immunother (2001) 50(3), 141-150) or pSPORT1 (GIBCO BRL).

In some embodiments, the expression control sequences are eukaryotic promoter systems in vectors capable of transforming of transfecting eukaryotic host cells, but control sequences for prokaryotic hosts may also be used. Once the vector has been incorporated into the appropriate host, the host is maintained under conditions suitable for high level expression of the nucleotide sequences, and as desired, the collection and purification of the polypeptide of the disclosure may follow.

Additional regulatory elements may include transcriptional as well as translational enhancers. Advantageously, the above-described vectors of the disclosure comprises a selectable and/or scorable marker. Selectable marker genes useful for the selection of transformed cells are well known to those skilled in the art and comprise, for example, antimetabolite resistance as the basis of selection for dhfr, which confers resistance to methotrexate (Reiss, Plant Physiol. (Life-Sci. Adv.) 13 (1994), 143-149); npt, which confers resistance to the aminoglycosides neomycin, kanamycin and paromycin (Herrera-Estrella, EMBO J. 2 (1983), 987-995) and hygro, which confers resistance to hygromycin (Marsh, Gene 32 (1984), 481-485). Additional selectable genes have been described, namely trpB, which allows cells to utilize indole in place of tryptophan; hisD, which allows cells to utilize histinol in place of histidine (Hartman, Proc. Natl. Acad. Sci. USA 85 (1988), 8047); mannose-6-phosphate isomerase which allows cells to utilize mannose (WO 94/20627) and ODC (ornithine decarboxylase) which confers resistance to the ornithine decarboxylase inhibitor, 2-(difluoromethyl)-DL-ornithine, DFMO (McConlogue, 1987, In: Current Communications in Molecular Biology, Cold Spring Harbor Laboratory ed.) or deaminase from Aspergillus terreus which confers resistance to Blasticidin S (Tamura, Biosci. Biotechnol. Biochem. 59 (1995), 2336-2338).

Useful scorable markers are also known to those skilled in the art and are commercially available. Advantageously, said marker is a gene encoding luciferase (Giacomin, Pl. Sci. 116 (1996), 59-72; Scikantha, J. Bact. 178 (1996), 121), green fluorescent protein (Gerdes, FEBS Lett. 389 (1996), 44-47) or β-glucuronidase (Jefferson, EMBO J. 6 (1987), 3901-3907). This embodiment is particularly useful for simple and rapid screening of cells, tissues and organisms containing a recited vector.

As described above, the recited oncolytic viral vector nucleic acid molecule can be used alone for cancer therapy. The vectors containing the DNA sequence(s) encoding any one of the above described bi-specific single chain antibody constructs is delivered to subjects that in turn produce the polypeptide of interest.

In accordance with the above, the present disclosure relates to methods to derive oncolytic viral vectors used conventionally in genetic engineering that comprise a nucleic acid molecule encoding the polypeptide sequence of a bi-specific single chain antibody constructs defined herein. Preferably, the oncolytic vector is an expression vector and/or a gene transfer or targeting vector. Methods that are well known to those skilled in the art can be used to construct recombinant vectors; see, for example, the techniques described in Sambrook et al. (loc cit.), Ausubel (1989, loc cit.) or other standard text books. The oncolytic viralvectors containing the nucleic acid molecules of the disclosure can be transferred into the host cell by well-known methods, which vary depending on the type of cellular host. For example, calcium phosphate treatment or electroporation may be used for cellular hosts; see Sambrook, supra.

It is further envisaged that the pharmaceutical composition of the disclosure comprises a host transformed or transfected with an oncolytic viral vector defined herein above. The host may be produced by introducing at least one of the above described vectors or at least one of the above described nucleic acid molecules into the host. The presence of the at least one vector or at least one nucleic acid molecule in the host may mediate the expression of a gene encoding the above described be specific single chain antibody constructs.

The described nucleic acid molecule or vector that is introduced in the host may either integrate into the genome of the host or it may be maintained extrachromosomally.

The host can be any eukaryotic cell or prokaryotic cell. It is particularly envisaged that the recited host may be a mammalian cell. Particularly preferred host cells comprise CV-1, BS-C-1, HuTK-143B, BHK-21, CEF, CHO cells, COS cells, myeloma cell lines like SP2/0 or NS/0 cells.

The pharmaceutical composition of the disclosure may also comprise a proteinaceous compound capable of providing an activation signal for immune effector cells useful for cell proliferation or cell stimulation. The proteinaceous compound is not understood as an additional domain of the above defined bispecific single chain antibody construct, but at least one additional component of the pharmaceutical composition of the disclosure.

In the light of the present disclosure, the “proteinaceous compounds” providing an activation signal for immune effector cells” may be, e.g. a further activation signal for T cells (e.g. a further costimulatory molecule: molecules of the B7-family, Ox40 L, 4.1 BBL), or a further cytokine: interleukin (e.g. IL-2), or an NKG-2D engaging compound. Preferred formats of proteinaceous compounds comprise additional bispecific antibodies and fragments or derivatives thereof, e.g. bispecific scFv. Proteinaceous compounds can comprise, but are not limited to scFv fragments specific for the T cell receptor or superantigens. Superantigens directly bind to certain subfamilies of T cell receptor variable regions in an MHC-independent manner thus mediating the primary T cell activation signal. The proteinaceous compound may also provide an activation signal for immune effector cell which is a non-T cell. Examples for immune effector cells which are non-T cells comprise, inter alia, NK cells.

One embodiment of the disclosure relates to a process for the production of a composition of the disclosure, the process comprising culturing a host defined herein above under conditions allowing the expression of the construct and recovering the produced bi-specific single chain antibody construct from the culture.

An embodiment of the disclosure relates to the use of a bi-specific single chain antibody construct as defined above, a nucleic acid sequence as defined above, a vector as defined above, a host as defined above and/or produced by a process as defined above for the preparation of a pharmaceutical composition for the prevention, treatment or amelioration of a cancerous disease, such as a tumorous disease. In particular, the pharmaceutical composition of the present disclosure may be particularly useful in preventing, ameliorating and/or treating cancer, including cancer having solid tumors, for example.

It is envisaged by the present disclosure that the above defined cells, bi-specific single chain antibody construct, nucleic acid molecules and vectors are administered either alone or in any combination using standard vectors and/or gene delivery systems, and in at least some cases together with a pharmaceutically acceptable carrier or excipient. Subsequent to administration, said nucleic acid molecules or vectors may be stably integrated into the genome of the subject.

In specific embodiments, viral vectors may be used that are specific for certain cells or tissues and persist in said cells. Suitable pharmaceutical carriers and excipients are well known in the art. The compositions prepared according to the disclosure can be used for the prevention or treatment or delaying the above identified diseases.

Furthermore the disclosure relates to a method for the prevention, treatment or amelioration of a tumorous disease comprising the step of administering to a subject in the need thereof an effective amount of cells harboring a bi-specific single chain antibody construct as defined above, a nucleic acid sequence as defined above, a vector as defined as defined above, and/or produced in by a process as defined above.

Embodiments of the present disclosure relate to a kit comprising a bispecific single chain antibody construct as defined above, a nucleic acid sequence as defined above, a vector as defined above and/or a host as defined above. It is also envisaged that the kit of this disclosure comprises a pharmaceutical composition as described herein above, either alone or in combination with further medicaments to be administered to an individual in need of medical treatment or intervention.

### III. THERAPEUTIC USES OF ONCOLYTIC VIRUSES

By way of illustration, cancer patients or patients susceptible to cancer or suspected of having cancer may be treated as described herein. Oncolytic viruses as described herein may be administered to the individual and retained for extended periods of time. The individual may receive one or more administrations of the viruses. In some embodiments, the viruses are encapsulated to inhibit immune recognition and placed at the site of a tumor. Embodiments of the disclosure further encompass a process for the production of the composition of the disclosure, a method for the prevention, treatment or amelioration of cancer, and the use of an oncolytic virus in the prevention, treatment or amelioration of cancer.

In various embodiments the expression constructs, nucleic acid sequences, vectors, host cells and/or pharmaceutical compositions comprising the same are used for the prevention, treatment or amelioration of a cancerous disease, such as a tumorous disease. In particular embodiments, the pharmaceutical composition of the present disclosure may be particularly useful in preventing, ameliorating and/or treating cancer, including cancer having solid tumors, for example.

Possible indications for administration of the composition(s) of the disclosure are cancerous diseases, including tumorous diseases, including breast, prostate, lung, and colon cancers or epithelial cancers/carcinomas such as breast cancer, colon cancer, prostate cancer, head and neck cancer, skin cancer, cancers of the genito-urinary tract, e.g. ovarian cancer, endometrial cancer, cervical cancer and kidney cancer, lung cancer, gastric cancer, cancer of the small intestine, liver cancer, pancreas cancer, gall bladder cancer, cancers of the bile duct, esophagus cancer, cancer of the salivatory glands and cancer of the thyroid gland. In specific embodiments, hematological malignancies including, but not limited to, leukemia, lymphoma, multiple myeloma, and myelodysplastic syndromes, are treated with methods and compositions of the disclosure. In specific embodiments, the cancer is EphA2-, HER2- GD2- or Glypican-3-positive, for example. In other specific embodiments, the cancer is positive for, e.g., displays on its cell surface, any of the tumor associated antigens or tumor specific antigens listed herein. The administration of the composition(s) of the disclosure is useful for all stages and types of cancer, including for minimal residual disease, early solid tumor, advanced solid tumor and/or metastatic solid tumor.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

In particular embodiments, the present invention contemplates, in part, viruses, expression constructs, nucleic acid molecules and/or vectors that can administered either alone or in any combination with another therapy, and in at least some aspects, together with a pharmaceutically acceptable carrier or excipient. In certain embodiments, prior to administration of the viruses, they may be combined with suitable pharmaceutical carriers and excipients that are well known in the art. The compositions prepared according to the disclosure can be used for the prevention or treatment or delaying of onset or worsening of cancer.

Furthermore, the disclosure relates to a method for the prevention, treatment or amelioration of a cancerous (including tumorous) disease comprising the step of administering to a subject in need thereof an effective amount of oncolytic viruses of the disclosure, wherein the virus expresses a molecule comprising an activation domain that binds to a target on an immune cell and an antigen recognition domain that binds one or more molecules produced by or present on a target cell. The disclosure includes nucleic acid sequence that encodes an engager, vector(s) that encodes an engager, as contemplated herein and/or produced by a process as contemplated herein.

The disclosure further encompasses co-administration protocols with other cancer therapies, e.g. bispecific antibody constructs, targeted toxins or other compounds, including those which act via immune cells, including T-cell therapy. The clinical regimen for co-administration of the inventive composition(s) may encompass co-administration at the same time, before and/or after the administration of the other component. Particular combination therapies include chemotherapy, radiation, surgery, hormone therapy, and/or other types of immunotherapy.

Embodiments relate to a kit comprising one or more oncolytic viruses as described herein, a nucleic acid sequence as described herein, a vector as described herein and/or a host as described herein. It is also contemplated that the kit of this disclosure comprises a pharmaceutical composition as described herein above, either alone or in combination with further medicaments to be administered to an individual in need of medical treatment or intervention.

The viruses may be introduced into a host organism, e.g., a mammal, in a wide variety of ways. The viruses may be introduced at the site of the tumor, in specific embodiments, although in alternative embodiments viruses hone to the cancer or are modified to hone to the cancer. The number of viruses that are employed will depend upon a number of circumstances, the purpose for the introduction, the lifetime of the cells, the protocol to be used, for example, the number of administrations, the stability of the viruses, and the like. The viruses may be applied in a dispersion, and may be injected at or near the site of interest. The viruses may be in a physiologically-acceptable medium.

The oncolytic virus may be administered intravenously or intraarterially. The oncolytic virus may be dispersed in a pharmaceutically acceptable formulation for injection. The individual may be administered between about 10⁵, 10⁶, 10⁷, 10⁸, 10⁹, 10¹⁰, 10¹¹, 10¹², 10¹³ pfu of virus. The individual may be administered the oncolytic virus multiple times, including 1, 2, 3, 4, 5, 6, or more times.

It should be appreciated that the system is subject to variables. Therefore, it is expected that for each individual patient, even if there were viruses that could be administered to the population at large, each patient would be monitored for the proper dosage for the individual, and such practices of monitoring a patient are routine in the art.

### IV. PHARMACEUTICAL COMPOSITIONS

In accordance with this disclosure, the term “pharmaceutical composition” relates to a composition for administration to an individual. In a preferred embodiment, the pharmaceutical composition comprises a composition for parenteral, transdermal, intraluminal, intra-arterial, intrathecal or intravenous administration or for direct injection into a cancer. It is in particular envisaged that said pharmaceutical composition is administered to the individual via infusion or injection. Administration of the suitable compositions may be effected by different ways, e.g., by intravenous, subcutaneous, intraperitoneal, intramuscular, topical or intradermal administration. The pharmaceutical composition of the present disclosure may further comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, etc. Compositions comprising such carriers can be formulated by well-known conventional methods. These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The compositions of the disclosure may be administered locally or systematically. Administration will generally be parenteral, e.g., intravenous; DNA may also be administered directly to the target site, e.g., by biolistic delivery to an internal or external target site or by catheter to a site in an artery. In a preferred embodiment, the pharmaceutical composition is administered subcutaneously and in an even more preferred embodiment intravenously. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the pharmaceutical composition of the present disclosure might comprise proteinaceous carriers, like, e.g., serum albumine or immunoglobuline, preferably of human origin. It is envisaged that the pharmaceutical composition of the disclosure might comprise, in addition to the proteinaceous bispecific single chain antibody constructs or nucleic acid molecules or vectors encoding the same (as described in this disclosure), further biologically active agents, depending on the intended use of the pharmaceutical composition.

### V. ONCOLYTIC VIRUSES

Oncolytic vaccinia viruses are promising anti-cancer agents owing to their ability to infect, replicate in, and lyse tumor cells, and spread to other tumor cells in successive rounds of replication. Oncolytic vaccinia virus therapy has shown promise in preclinical models and in clinical studies. However, the antitumor efficacy of the oncolytic vaccinia virus is suboptimal. This is most likely due to the limited virus spread through the tumor and limited activation of antitumor T-cell responses within the tumor of oncolytic vaccinia virus. Therefore, the present disclosure provides an oncolytic vaccinia virus that 1) facilitates T-cell tumor infiltration and activation, and 2) effectively lyses tumor cells that are not infected the vaccinia virus (by-stander killing). These aspects overcome the current limitations of oncolytic vaccinia viruses.

Oncolytic viruses may utilize DNA or RNA as their genetic material. Oncolytic DNA viruses may have capsid symmetry that is isocahedral or complex. Isosahedral oncolytic DNA viruses may be naked or comprise an envelope. Families of oncolytic DNA viruses include the Adenoviridae (for example, Adenovirus, having a genome size of 36-38 kb), Herpesviridae (for example, HSV1, having a genome size of 120-200 kb) and Poxviridae (for example, Vaccinia virus and myxoma virus, having a genome size of 130-280 kb). Oncolytic RNA viruses include those having icosahedral or helical capsid symmetry. Icosahedral oncolytic viuses are naked without envelope and include Reoviridae (for example, Reovirus, having a genome of 22-27 kb) and Picornaviridae (for example, Poliovirus, having a genome size of 7.2-8.4 kb). Helical oncolytic RNA viruses are enveloped and include Rhabdoviridae (for example, VSV, having genome size of 13-16 kb) and Paramyxoviridae (for example MV and NDV, having genome sizes of 16-20 kb).

Any of these types of oncolytic viruses may be employed in the disclosure. In specific embodiments, Vaccinia virus is employed.

### VI. KITS

Any of the compositions described herein may be comprised in a kit. In a non-limiting example, one or more viruses and/or the reagents to generate or manipulate the virus may be comprised in a kit. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile, pharmaceutically acceptable buffer and/or other diluent.

In particular embodiments, viruses for use in therapy are provided in a kit, and in some cases the viruses are essentially the sole component of the kit. The kit may comprise reagents and materials to modify the desired virus. In specific embodiments, the reagents and materials include expression constructs, primers for amplifying desired sequences, restriction enzymes, one or more DNAs for inclusion in the virus, nucleotides, suitable buffers or buffer reagents, salt, and so forth, and in some cases the reagents include vectors and/or DNA that encodes an engager molecule as described herein and/or regulatory elements therefor.

In particular embodiments, there are one or more apparatuses in the kit suitable for extracting one or more samples from an individual. The apparatus may be a syringe, scalpel, and so forth.

In some cases, the kit, in addition to the virus embodiments, also includes a second cancer therapy, such as chemotherapy, hormone therapy, and/or immunotherapy, for example. The kit(s) may be tailored to a particular cancer for an individual and comprise respective second cancer therapies for the individual.

### VII. EXAMPLES

The following examples are presented in order to more fully illustrate the preferred embodiments of the disclosure. They should in no way, however, be construed as limiting the broad scope of the disclosure.

### Example 1

### T-Cell Engager-Armed Oncolytic Virus (TEA-OV) as a Novel Cancer Therapy

The significance of at least certain embodiments of the present disclosure is that the development of an improved oncolytic vaccinia virus with the unique ability of inducing bystander killing of tumor cells that are not infected with vaccinia virus, resulting in enhanced antitumor effects. Established herein is the principle of arming oncolytic vaccinia virus with a T-cell engager as a novel oncolytic vaccinia virus therapy, thereby opening a new avenue for development of effective oncolytic virus therapy. The oncolytic vaccinia virus of embodiments of the present disclosure represents the first example of enhancing oncolytic vaccinia virus by incorporating a T-cell engager. This new strategy is applicable to improving the efficacy of various forms of oncolytic virus, such as oncolytic adenovirus (AdV), herpes simplex virus (HSV), reovirus, myxoma virus (MYXV), poliovirus, vesicular stomatitis virus (VSV), measles virus (MV), and Newcastle disease virus (NDV), for example.

Importantly, embodiments of the present disclosure provide a therapeutic composition that is useful for the treatment of advanced solid tumors. The following examples evaluate the efficacy of new oncolytic vaccinia virus in human tumor xenograft models. The TEA-VV embodiments are useful for treatment of at least advanced solid tumors, including those that are often incurable with current treatment strategies, for example.

In particular aspects, TEA-OV technology is highly innovative because it tested if arming oncolytic vaccinia virus with a T-cell engager CD3-scFv results in enhanced antitumor effects. In specific embodiments, TEA-OV exerts its anti-tumor activity through one or two mechanisms: i) bi-specific scFv direct T cells to recognize and kill tumor cells that are not infected with vaccinia virus (by-stander killing), resulting in enhanced tumor lysis, and ii) CD3-scFv activate T-cells within the tumor, and the cytokines they release upon activation create a pro-inflammatory microenvironment, inhibiting tumor growth.

### Example 2

### Development of a T-Cell Engager-Armed Oncolytic Vaccinia Virus (TEA-VV) for Advanced Solid Tumors

There is increasing evidence that T cells are able to control tumor growth and survival in cancer patients, both in early and late stages of the disease. For example, adoptive transfer of T cells has been demonstrated to effectively treat disseminated tumors including Hodgkin's Lymphoma, nasopharyngeal carcinoma, neuroblastoma and melanoma. However, tumor-specific T-cell responses are difficult to mount and sustain in cancer patients and are limited by numerous immune escape mechanisms of tumor cells selected during immunoediting. Therefore, it would be desirable to develop an alternative strategy to engage T cells for cancer therapy with the ability of overcoming the immune escape mechanism of tumors. The present disclosure provides such a strategy.

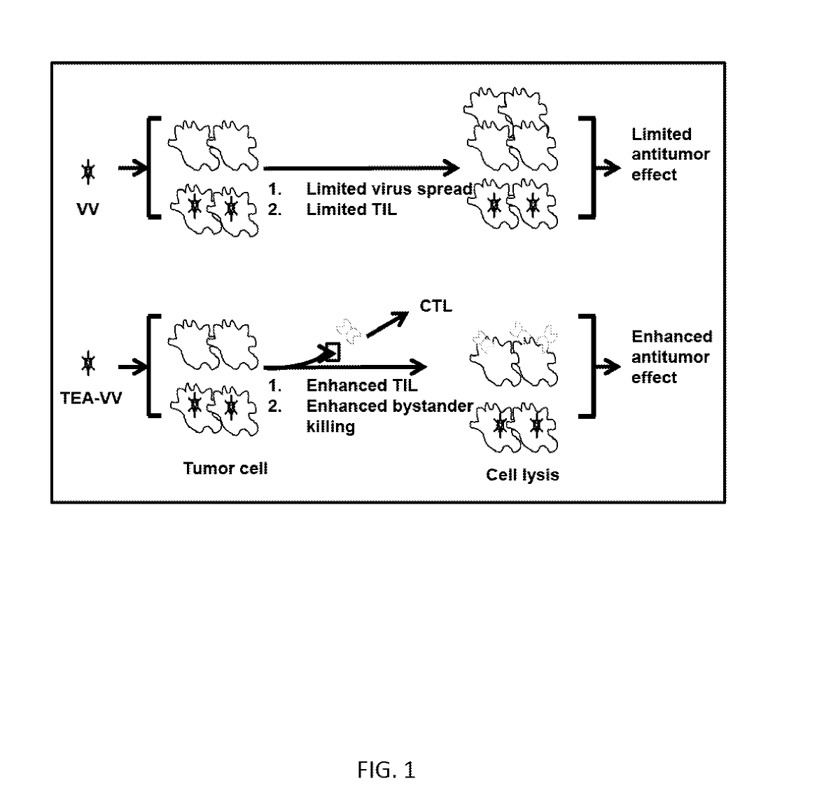

Arming oncolytic vaccinia virus with T-cell engager CD3-scFv is a useful means of engaging T cells for cancer therapy. CD3-scFv has been used in Bi-specific T-cell engagers (BiTE) that consists of CD3-scFv and a scFv specific for a tumor surface antigen. Clinical studies have shown that BiTE therapy results in highly effective killing of tumor cells in patients with non-Hodgkin's lymphomas and B-cell precursor acute lymphoblastic leukemia. In the present disclosure, there is a T-cell engager CD3-scFv armed oncolytic vaccinia virus (TEA-VV). TEA-VV encodes bi-specific scFvs that will direct T cells to recognize and kill tumor cells that are not infected with vaccinia virus (by-stander killing), resulting in enhanced tumor lysis. In addition, the CD3-scFv will promote T-cell infiltration into tumors and their activation, and the cytokines they release upon activation will create a pro-inflammatory microenvironment, inhibiting tumor growth. Importantly, CD3-scFvs are capable of redirecting the vast number of existing T-cell clones in patients to tumor cells, thus overcoming many of the immune escape mechanisms of tumors. In addition, oncolytic virus induces local production of T-cell engager that might allow higher concentrations at the target while reducing systemic side effects. Thus arming oncolytic vaccinia virus with bi-specific scFvs provides an alternative approach to engage T cells for cancer therapy and produce the desired increase in antitumor activity of current vaccinia virus therapy by inducing by-stander killing (FIG. 1).

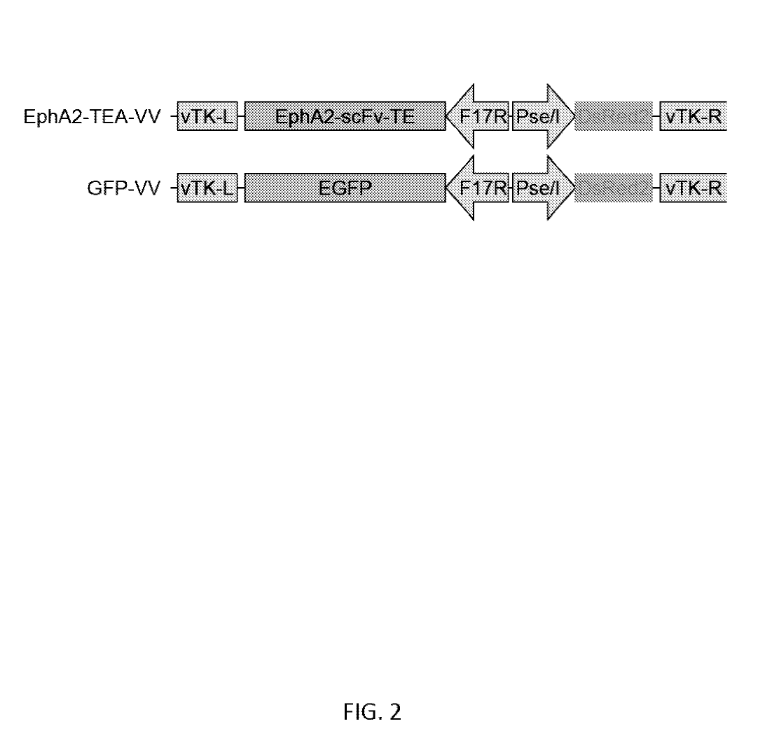

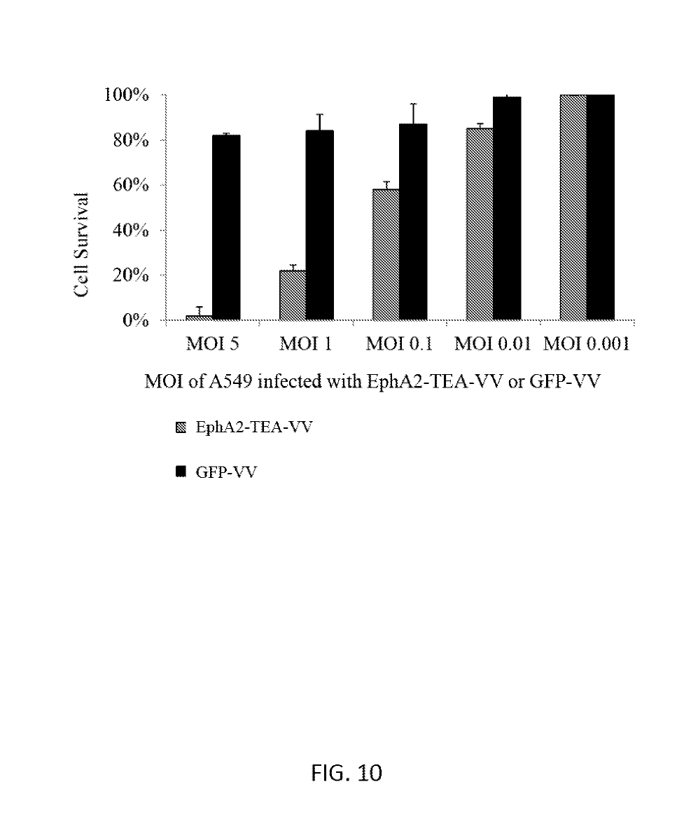

There is developed a CD3-scFv armed vaccinia virus as an effective virus therapy approach for advanced solid tumors. In particular embodiments of the disclosure, oncolytic vaccinia virus expressing bi-specific scFvs bind both to CD3 and a tumor cell antigen will promote T-cell infiltration into tumors and by-stander killing of tumor cells that are not infected with vaccinia virus, resulting in enhanced antitumor activity. In the studies, human CD3-scFvs expressed as secretory bi-specific scFvs were produced that bind to CD3 and a tumor cell antigen EphA2 (EphA2-TEA-VV) (FIG. 2). The human lung cancer cell line A549 expressing secretory EphA2-CD3-scFv induced killing of EphA2-positive A549 that are not infected with virus, suggesting the induction of bystander tumor killing (FIG. 10). In an exemplary embodiment (FIG. 2), EphA2-TEA-VV comprises one molecule comprising the F17R promoter regulating bi-specific EphA2-scFv-CD3-scFv and Psel promoter regulating a label, such as DsReds; alternative configurations of the components may also be suitable.

The present disclosure, in some embodiments, provides an effective oncolytic VV for the treatment of advanced solid tumors. This disclosure has established the principle of arming oncolytic VV with a T-cell engager as a novel oncolytic VV, thereby opening a new avenue for the development and use of improved oncolytic virus therapy. This strategy is applicable to various forms of oncolytic virus.

### Example 3

### Construction of EphA2-TEA-VV

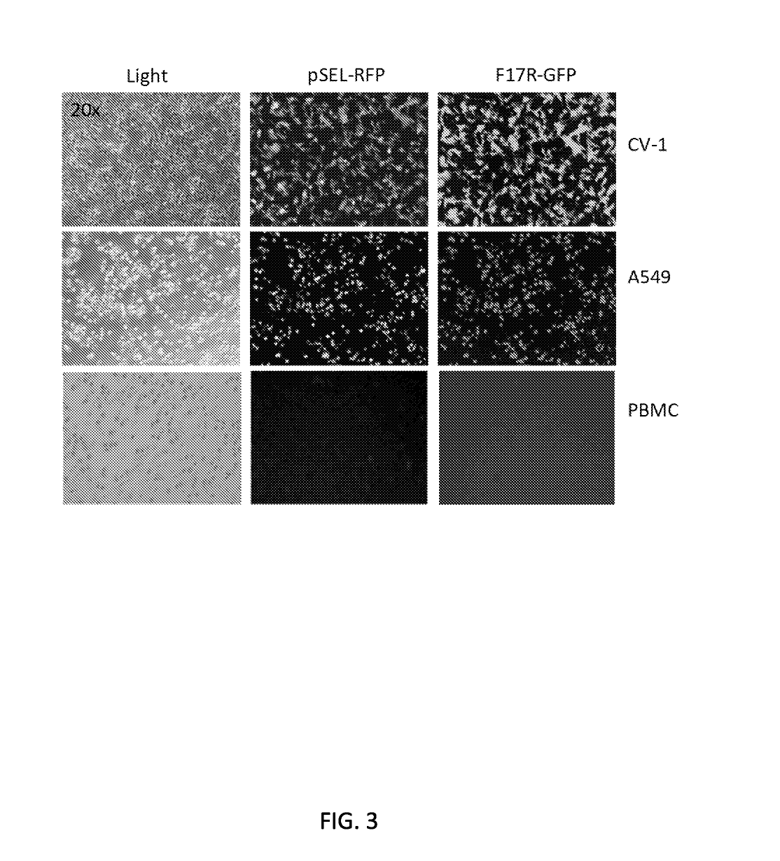

Vaccinia viruses (Western Reserve strain) were generated expressing secretory EphA2-scFv-CD3-scFv (EphA2-TEA-VV) or GFP (GFP-VV) by recombination of a version of pSEL shuttle plasmid containing T-cell engagers into the TK gene of the vSC20 strain of WR vaccinia virus. Firstly, the shuttle vector pSEL was constructed to contain the EphA2-scFv-CD3-scFv, or GFP (FIG. 2). The inserted T-cell engagers were expressed under the transcriptional control of the F17R late promoter to allow for sufficient viral replication before T-cell activation. The VVs also express DsRed2 to allow for monitoring infectivity (FIG. 3). For constructing the recombinant virus encoding TEs, the shuttle vectors pSEL were transfected into human 143 TK− cells. Cells were then infected with virus VSC20 at a multiplicity of infection (MOI) of 0.1. After three rounds of plaque selection and amplification with confirmation of the expression of TEAs, one of the clones was selected for amplification and purification.

### Example 4

### EphA2-TEA-VV Expressed Secretory Functional Bi-Specific EphA2-scFv-CD3-scFv

It was investigated if EphA2-TEA-VV-infected tumor cells express secretory bi-specific EphA2-scFv-CD3-scFv. To do this, the lung cancer cell line A549 was transduced with EphA2-TEA-VV or GFP-VV at MOI 5 and at 24 hours after incubation, cell culture medium were collected. Fresh human PBMC were then incubated with the medium collected from VV-infected A549 culture, followed by staining with EphA2-FITC (EphA2 protein labeled with FITC), CD8-PE, and CD4-APC. Flow analysis demonstrated that EphA2-TEA-VV-infected A549 expressed secretory bi-specific EphA2-scFv-CD3-scFv with the function to bind both to human T cells and EphA2 (FIG. 3). This result indicated that EphA2-TEA-VV-infected tumor cells expressed secretory bi-specific EphA2-scFv-CD3-scFv with the ability of binding both to human T cells and tumor antigen EphA2.

### Example 5

### EphA2-TE Expression does not Impair VVs Ability to Replicate

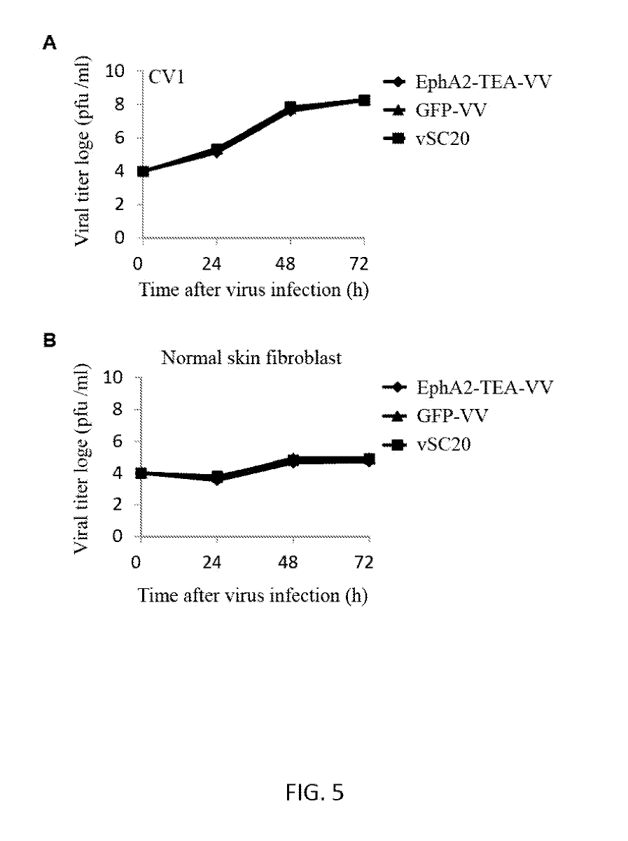

To demonstrate that EphA2-TEs do not impair the ability of EphA2-TEA-VV to replicate, CV-1 cells and normal human skin fibroblast were infected with EphA2-TEA-VV, GFP-VV and parental vSC20 VV. Infection of CV-1 with EphA2-TEA-VV, GFP-VV, or vSC20 yielded similar amounts of virus at various time points (FIG. 5). In contrast all three viruses replicated poorly in normal human skin fibroblasts (FIG. 5). Thus EphA2-TEs do not interfere with VV replication.

### Example 6

### EphA2-TE Expression does not Impair VVs Ability to Induce Tumor Cell Lysis

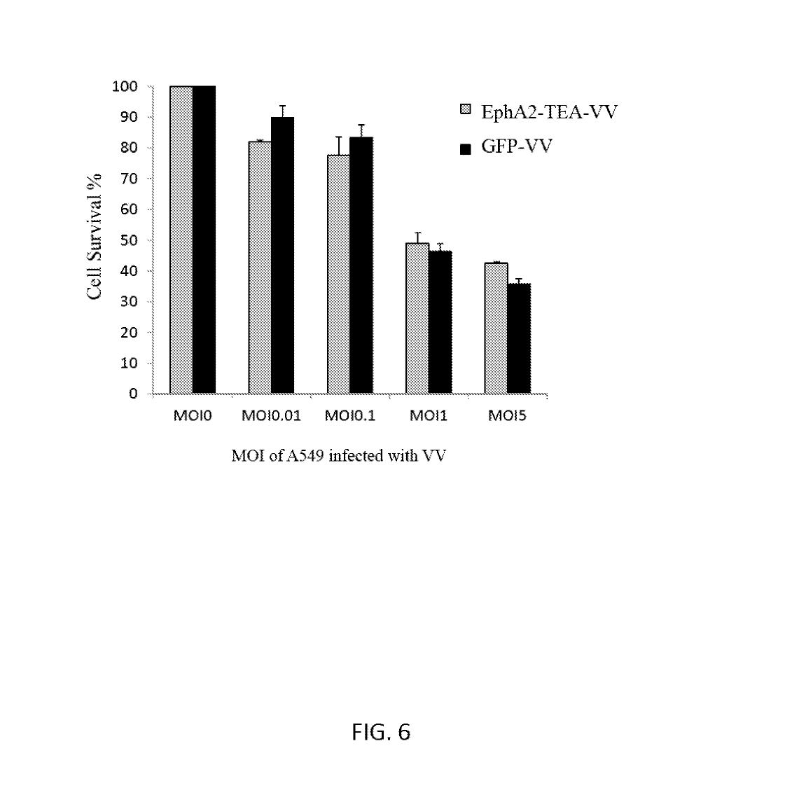

The ability of EphA2-TEA-VV or GFP-VV to induce tumor cell lysis in the absence of human T cells was compared. A549 cells were transduced with EphA2-TEA-VV or GFP-VV at increasing MOIs (0, 0.01, 0.1, 1, or 5), and 48 hours post virus infection, A549 viability was determined by MTS assay. A549 cells were killed with increasing MOI regardless of the used VV (FIG. 6). There was no difference between EphA2-TEA-VV and GFP-VV, indicating that the expression of EphA2-TE does not interfere with VV ability to induce tumor cell lysis.

### Example 7

### EphA2-TEA-VV Displayed Significantly Enhanced Tumor Lytic Activity Against A549 in the Presence of Human T Cells

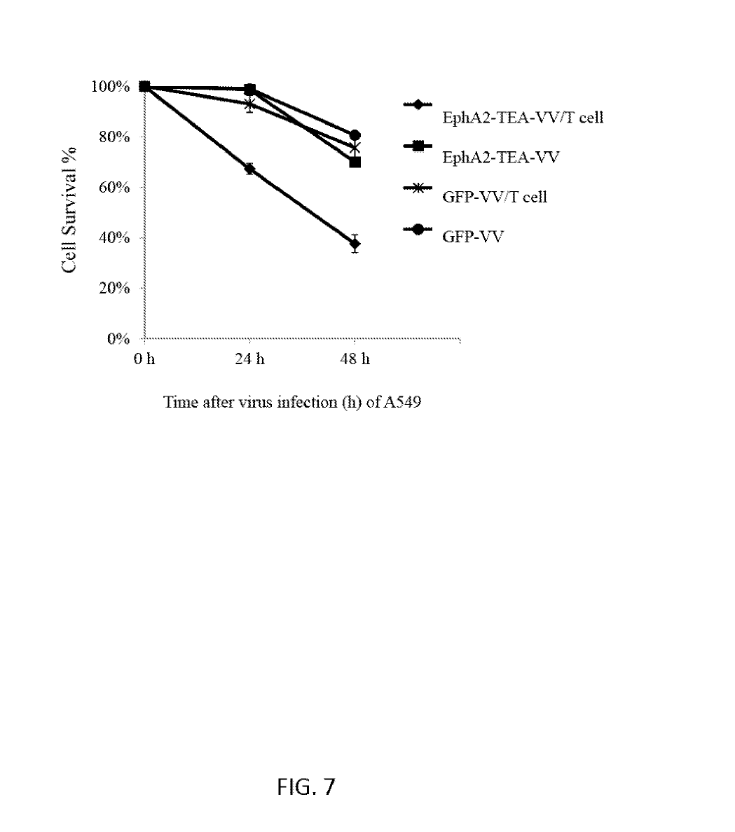

It was then determined if there was EphA2-TEA-VV's tumor lytic activity in the presence of human T cells. First, A549 cells were infected with EphA2-TEA-VV or GFP-VV at an MOI of 0.1. Human T cells were added as described above, and 24 or 48 hours post virus infection, A549 viability was determined by MTS assay. Only EphA2-TEA-VV displayed enhanced oncolytic activity in the presence of human T cells at 24 hr (EphA2-TEA-VV vs. GFP-VV, 75% vs. 100%) and 48 hr (EphA2-TEA-VV vs. GFP-VV, 35% vs 81%) (FIG. 7).

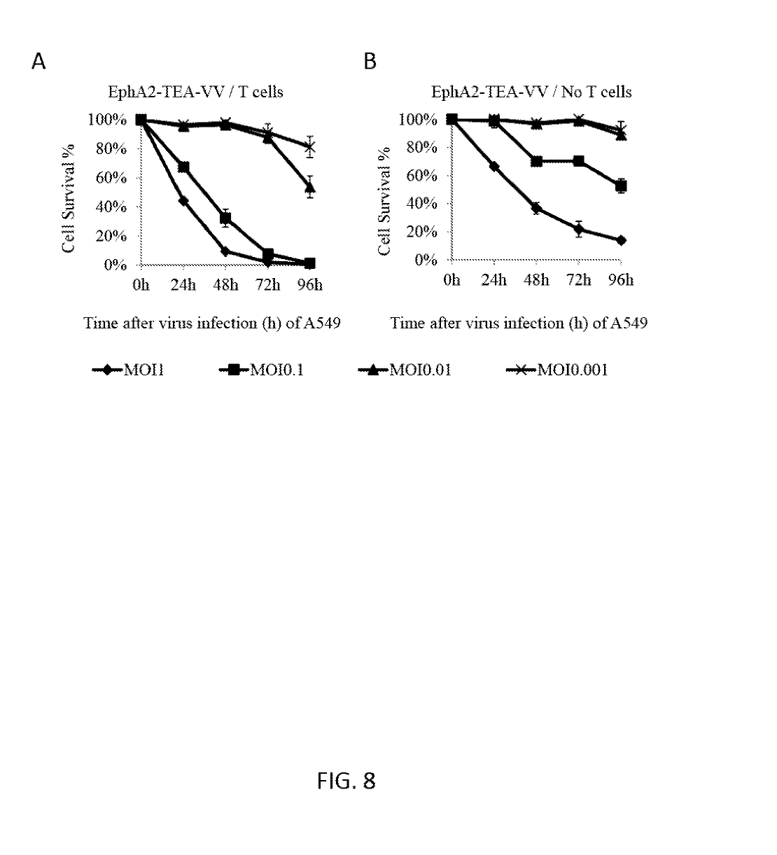

To further determine if EphA2-TEA-VVs redirect human T cells to A549 cells, cells were infected with EphA2-TEA-VV at increasing MOI (MOI 0.001, 0.01, 0.1, or 1). Next, human T cells, unstimulated T cells isolated from PBMCs with CD4/CD8 microbeads, were added to A549 cells at a T-cell to A549 ratio of 5:1. At 24, 48, 72, or 96 hours post virus infection, A549 viability was determined by MTS assay. A549 cells infected only with EphA2-TEA-VVs served as controls. EphA2-TEA-VV by itself induced cell killing in a dose dependent manner. However, even at the highest MOI tested, 15% of tumor cells were still alive 96 hours post infection (FIG. 8B). Adding human T cells to the culture significantly (p<0.05) increased anti tumor effects with all tumor cells being killed within 96 hours post infection at a MOI of 0.1 and 1 (FIG. 8A).

Taken together, the data indicates that EphA2-TEA-VVs redirect human T cells to A549 cells.

### Example 8

### EphA2-TEA-VVs Activate T Cells

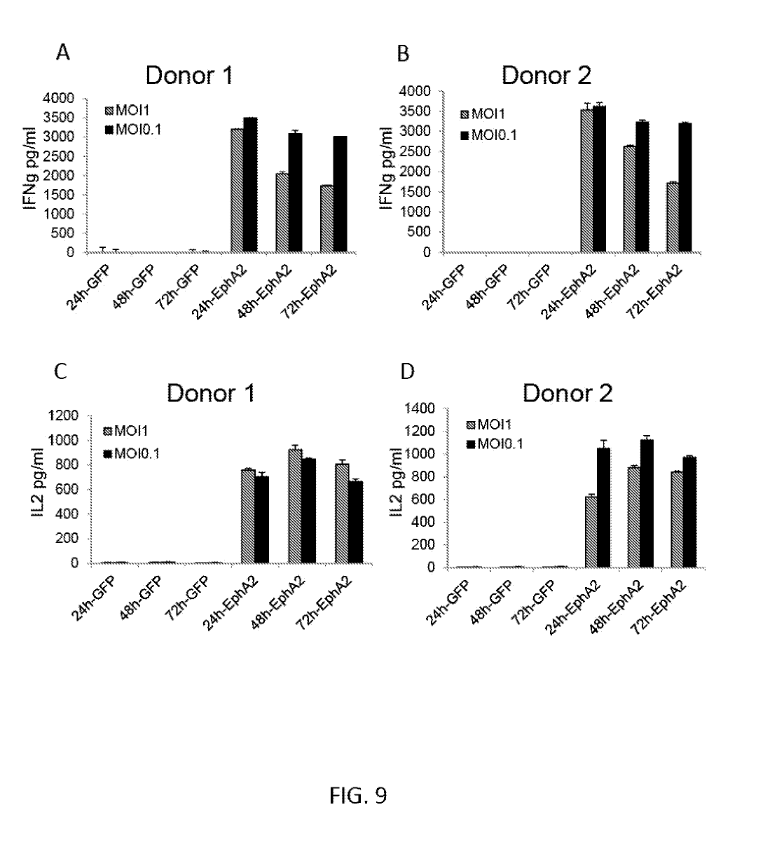

To determine if EphA2-TEs secreted by EphA2-TEA-VV not only redirect T cells to tumor cells but also activates human cells, A549 cells were infected with EphA2-TEA-VV or GFP-VV at an MOI of 1 or 0.1. Human T cells were added as described above, and 24 or 48 hours post virus infection, cell culture media was collected to determine the presence of pro-inflammatory cytokines by ELISA. Human T cells were activated by EphA2-TEs as evidenced by the production of proinflammatory cytokines such as IFN-beta and IL-2 in the cell culture supernatant of EphA2-TEA-VV infected A549 and human T cells, compared to that of GFP-VV infected A549 and human T cells (p<0.05). T cells produced little to no IFN-beta and IL-2 in response to GFP-VV infected A549 (FIG. 9). These results indicate that T-cell activation depends on the expression of EphA2-TEs by tumor cells.

### Example 9

### TEA-VV Induced by-Stander Killing of Non-Infected Tumor Cells

The ability of the TEA-VVs to induce by-stander killing of tumor cells that are not infected with vaccinia virus was considered. First, the EphA2-positive lung cancer cell line A549 was transduced with EphA2-TEA-VV, mTEA-VV, or GFP-VV at various MOI. The cell culture medium was collected after 48 hours culture. Then, non-infected A549 were co-cultured with fresh PBMC in the presence of the cell culture medium collected from the culture of TEA-VVs-infected A549. 48 hours later, tumor killing was measured using MTS assay. The results demonstrated that A549 was only killed in the presence of cell culture medium of EphA2-TEA-VV-infected A549, indicating the bystander killing of tumor cells (FIG. 9).

### Example 10

### EphA2 T Cell Engager in Vaccinia Virus Displays Enhanced Antitumor Effect

The effect of the exemplary EphA2-TEA-VV was characterized in its enhancement of antitumor effect in the presence of human T cells using an in vivo tumor model.

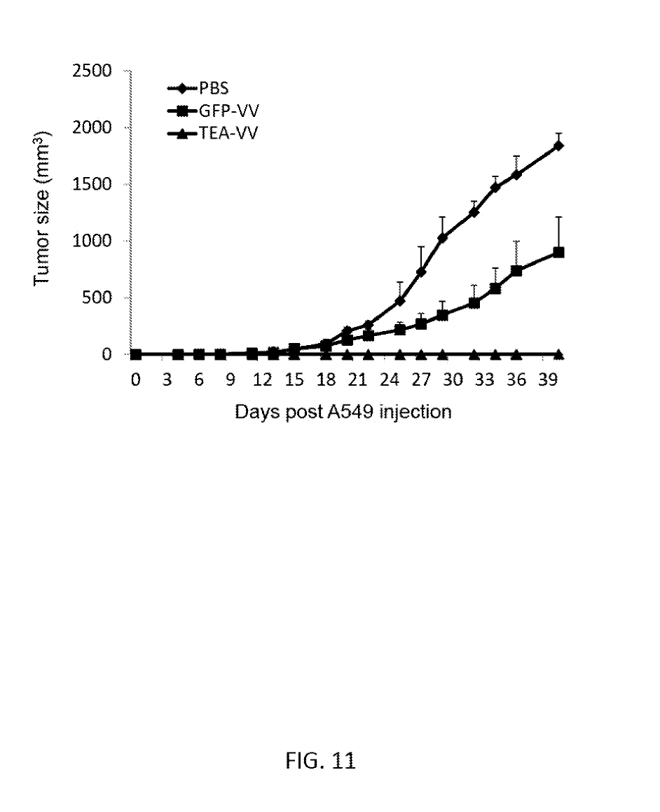

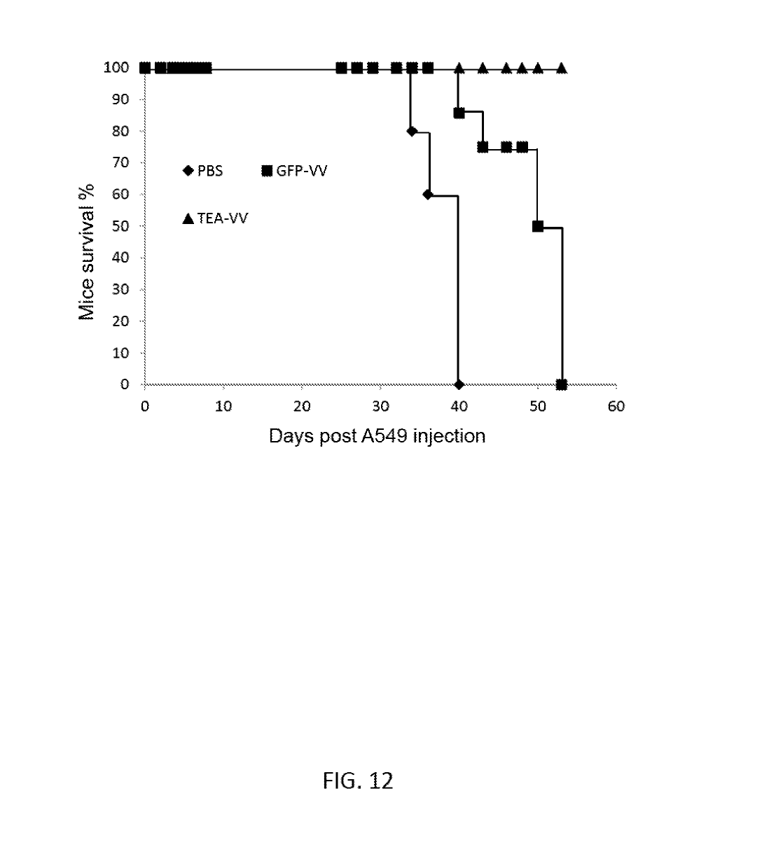

To investigate the anti-tumor effects of EphA2-TEA-VV in vivo, we initially used the s.c. A549 tumor model. To establish A549 tumors, 2×10⁶ A549 cells were mixed with unstimulated PBMC from healthy donor and inoculated subcutaneously into the right flank of SCID-Bg mice, followed by i.p. injection of 1×108 vp of EphA2-TEA-VV or GFP-VV on day 0. The mice that received PBS served as control. GFP-VVs moderately inhibited tumor growth compared to that in control mice. In contrast, mice that received EphA2-TEA-VV showed a significant decrease in tumor growth, compared to mice that received GFP-VV or PBS (EphA2-TEA-VV vs. GFP-VV p<0.0001) (FIG. 11). In addition, EphA2-TEA-VV also largely increased the mice survival compared to GFP-VV or PBS groups (FIG. 12).

To investigate the anti-tumor effects of EphA2-TEA-VV, luciferase-expressing A549 cells (A549.eGFP.ffluc) were injected i.v. into SCID-Bg mice on day 0. On day 7, an admixture of 10×106 untreated PBMCs and 1×10⁸ vp of EphA2-TEA-VV or GFP-VV were administered i.v. The mice that received only PBMCs served as control. Quantification of bioluminescent imaging showed that GFP-VVs plus PBMCs only moderately inhibited tumor growth compared to controls. In contrast, mice that received EphA2-TEA-VV plus PBCMs showed a significant decrease in tumor growth, compared to mice that received GFP-VV plus PBMCs or PBMCs alone (FIG. 12, EphA2-TEA-VV vs. GFP-VV p<0.05). Thus, in both animal models, EphA2-TEA-VV resulted in enhanced anti-tumor activity in comparison to GFP-VV.

### Example 11

### Construction and Function of HER2-TE

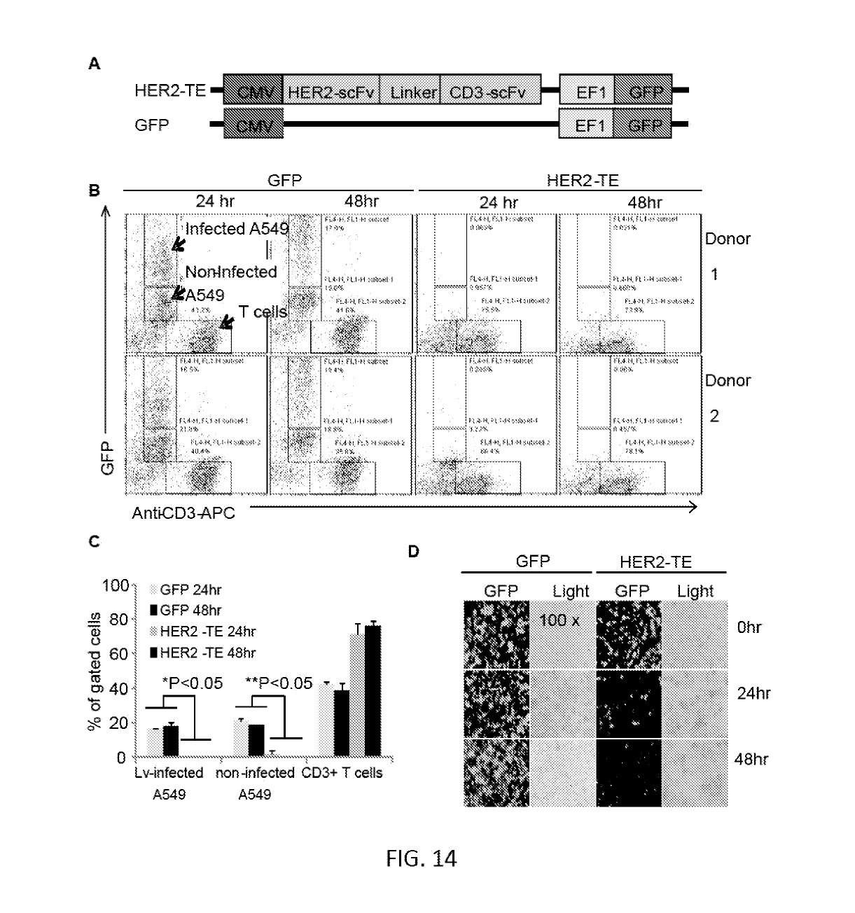

To characterize the ability of secretory bi-specific HER2-TE to induce killing of HER2+ tumor cells, lentivirus was constructed encoding secretory bi-specific HER2-TE and GFP (HER2-TE), or GFP only (GFP) (FIG. 14A). Lv-HER2-TE or Lv-GFP-infected A549 were mixed with non-infected A549 (GFP-), followed by co-culturing with unstimulated PBMC for two days. The tumor cell killing was measured by flow cytometry. Lv-HER2-TE induced killing of both GFP+ and GFP-A549 cells, while Lv-GFP didn't induce killing of A549, indicating that expression of secretory bi-specific TE by infected tumor cells induced by-stander killing of tumor cells that are not infected with the virus (FIG. 14B). Taken together, these results demonstrated that HER2-TE induced CD3+ T cells-mediated killing of non-infected A549 cells.

### Example 12

### Comparison of Bi-scFv Tandem HER2-TE and Diabody HER2-TE

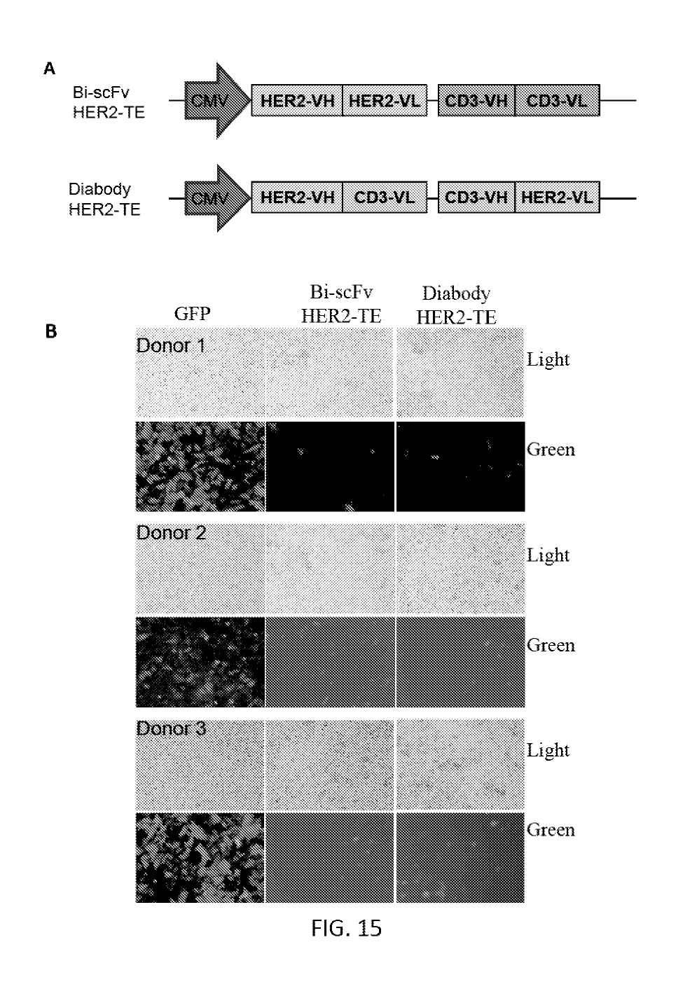

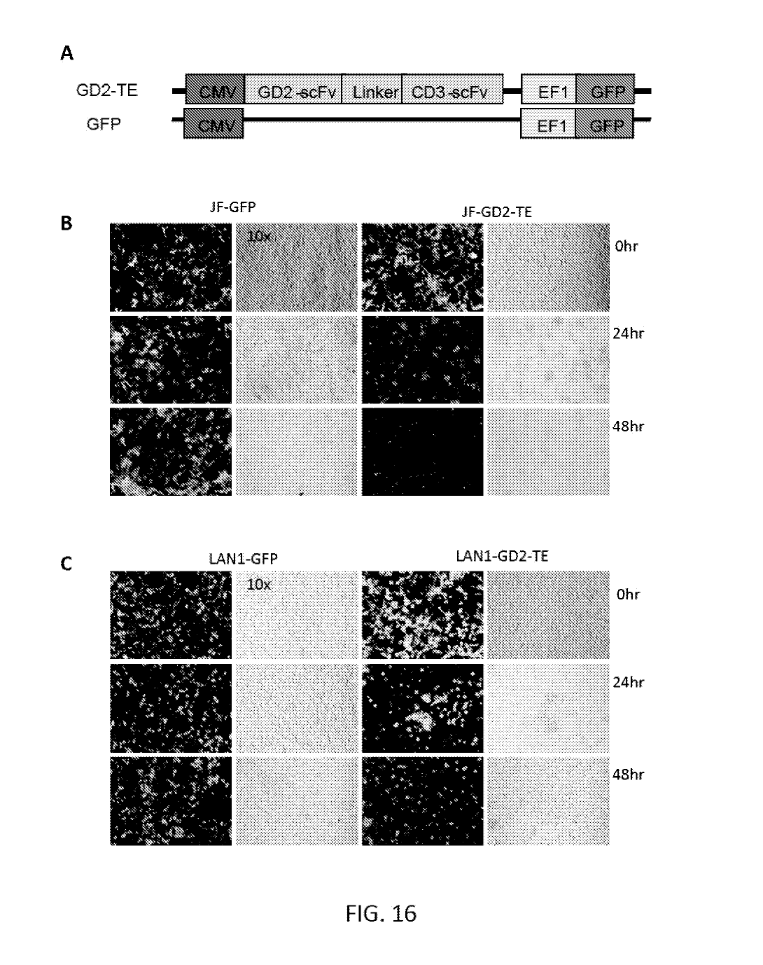

To compare the ability of bi-specific tandem HER2-TE and diabody HER2-TE to induce killing of GD2+ tumor cells, lentivirus was constructed encoding secretory bi-specific tandem HER2-TE (Bi-scFv-HER2-TE), diabody HER2-TE (Diabody-HER2-TE) or GFP only (GFP) (FIG. 15). Lv-Bi-scFv-HER2-TE, Lv-Diabody-HER2-TE, or Lv-GFP-infected HER2+ tumor cell line A549 were co-cultured with unstimulated PBMC. The tumor cell killing was monitored under the microscope after 24 h. Both Lv-Bi-scFv-HER2-TE or Lv-Diabody-HER2-TE induced killing of HER2+A549 tumor cell effectively, while Lv-GFP didn't induce killing of them (FIG. 16B). These results demonstrated that both bi-specific tandem HER2-TE and diabody HER2-TE induced CD3+ T cells-mediated killing of HER2+ tumor cells effectively.

### Example 13

### Construction and Function of GD2-TE

To characterize the ability of bi-specific GD2-TE to induce killing of GD2+ tumor cells, lentivirus was constructed encoding secretory bi-specific GD2-TE and GFP (GD2-TE), or GFP only (GFP) (FIG. 16A). Lv-GD2-TE or Lv-GFP-infected GD2+ tumor cell line JF or LAN2 were co-cultured with unstimulated PBMC. The tumor cell killing was monitored under the microscope. Lv-GD2-TE induced killing of both GD2+ tumor cell line JF and LAN1, while Lv-GFP didn't induce killing of them (FIG. 16B). These results demonstrated that GD2-TE induced CD3+ T cells-mediated killing of GD2+ tumor cells.

TEA-OV technology was developed to generate an enhanced oncolytic virus as cancer therapy. This technology can be adapted for any known oncolytic virus and any know tumor antigens. They have demonstrated—using oncolytic vaccinia virus and the EphA2 tumor antigen as a model—that oncolytic virus expressing T-cell engager i) kill EphA2-positive tumor cells, ii) induce bystander killing of EphA2-positive tumor cells that are not infected with virus. This technology has broad application for the entire field of virus cancer therapy and can be adapted to any oncolytic virus and tumor antigen of choice.

### Example 14

### Illustrative Examples

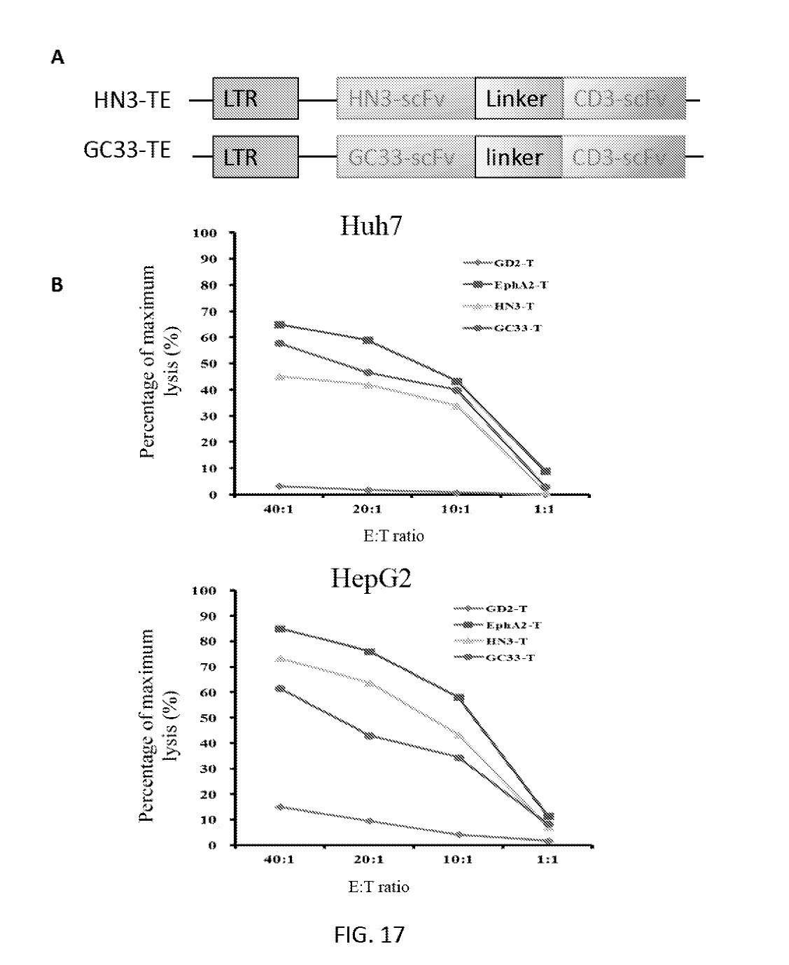

Construction and function of HN3-TE and GC33-TE for Hepatocellular carcinoma (HCC) (HN3 and GC33 are two antibody clones against HCC antigen Glypican-3). To characterize the ability of bi-specific EphA2-TE, HN3-TE and GC33-TE to induce killing of HCC, retrovirus (Rv) was constructed encoding secretory bi-specific EphA2-TE, HN3-TE, GC33-TE, and GD2-TE (FIG. 17A). Retrovirus infected human PBMC were co-cultured with HCC line Huh7 or HepG2 and tumor cytotoxicity was determined by 6 hours Cr release CTL assay. The results demonstrated that EphA2-TE, HN3-TE, or GC33-TE effectively induced lysis of HCC Huh7 or HepG2.

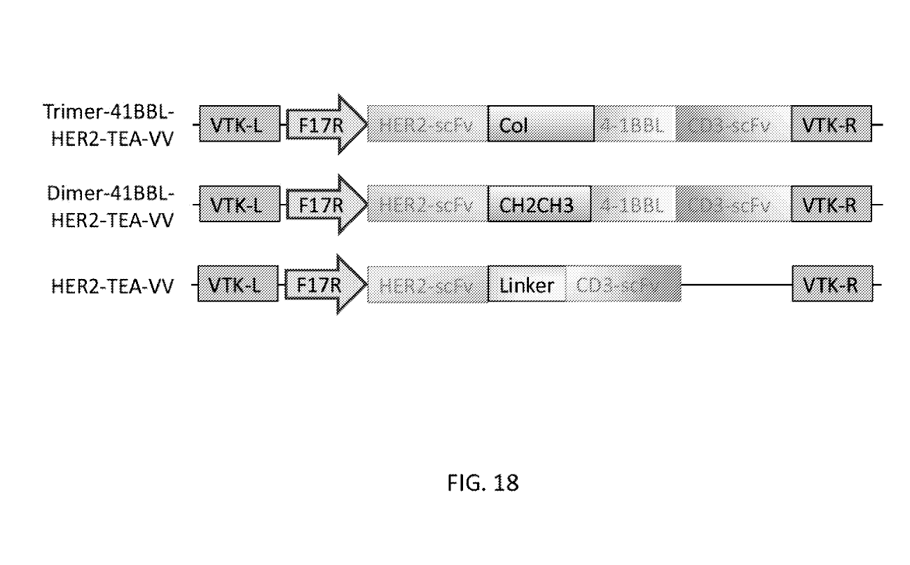

FIG. 18 shows trimer or dimer form of 4-1BBL-armed T cell engager were constucted. Trimer-41BBL-HER2-T-cell engager encodes an N-terminal trimerization region of collagen XV NC1 domain (col) that will form a stable trimer. Dimer-41BBL-HER2-T-cell engager encodes an human IgG1 CH2CH3 domain that will form a stable dimer. 4-1BBL belongs to the TNF superfamily and its dimeric or trimeric form is required for a significant costimulatory function.

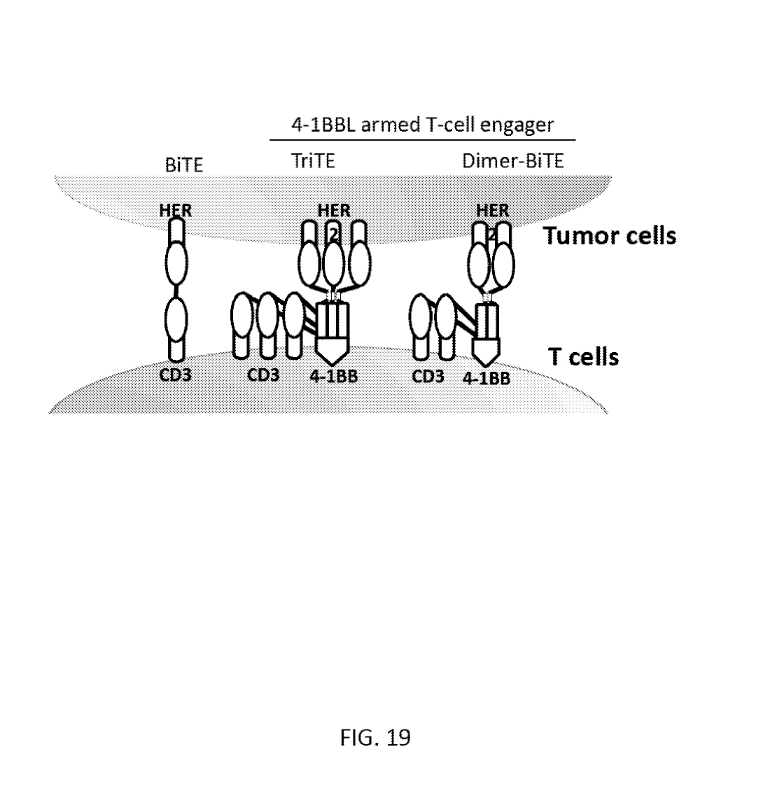

FIG. 19 illustrates enhanced antitumor effects of 4-1BBL-armed TEA-VV. 4-1BBL-armed TEA-VV enhances TEA-VV's antitumor effects through three mechanisms: 1) Trimerization or dimerization of T-cell engager increases binding affinity of scFv to target, 2) 41BB-L further activates T cells, and 3) 41BB-L co-stimulation endows T cells with the ability of overcoming immune suppression mediated by regulatory T cells.

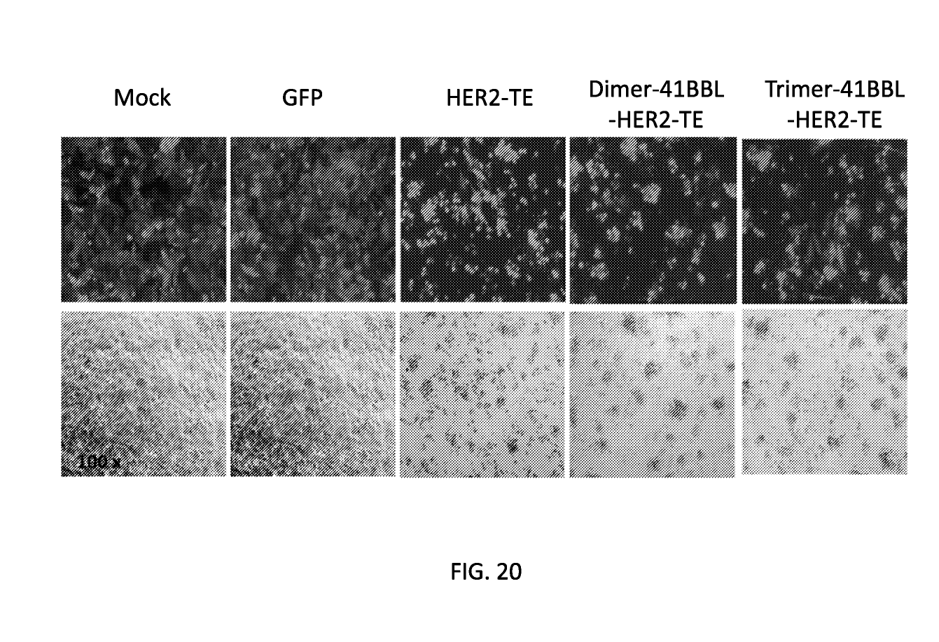

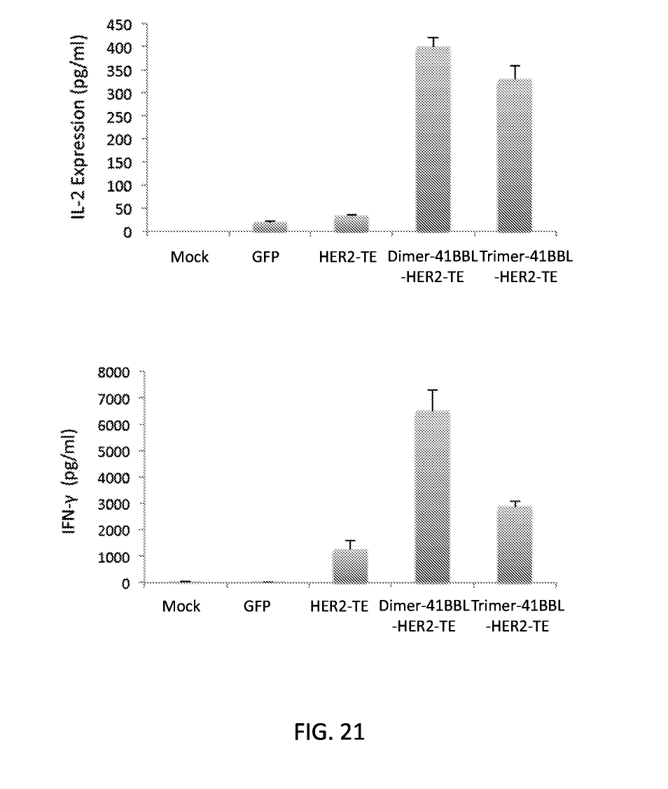

FIG. 20 shows that co-stimulatory signaling 4-1BBL further enhanced tumor killing effects and induced T cell activation. Lentivirus was generated expressing 41BBL-HER2-T cell engager or control lentiviral vectors (Lv-GFP, Lv-HER2-TE, Lv-dimer-41BBL-HER2-TE, or Trimer-41BBL-HER2-TE). Human A594-GFP lung tumor cells (HER2 positive) were infected with lentivirus and co-cultured with unstimulated human PBMC. 48 hours later, it was observed that costimulation of 4-1BBL significantly enhanced lytic activity of TEA-VVs. In addition, the cell culture supernatants were collected for cytokine ELISA. Unstimulated human PBMCs were activated by 41BBL as judged by the production of proinflammatory cytokines such as IFNγ and IL2 (FIG. 21).

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. An oncolytic virus that encodes a bipartite molecule comprising a single chain variable fragment (scFv) specific for a cell surface molecule and a scFv specific for a tumor antigen.

2. The virus of claim 1, wherein the cell surface molecule is on effector cells.

3. The virus of claim 2, wherein the effector cells are T lymphocytes.

4. The virus of claim 1, wherein the tumor antigen is selected from the group consisting of EphA2, HER2, or GD2.

5. The virus of claim 1, wherein the cell surface molecule is selected from the group consisting of CD3, CD4, CD5, CD8, CD16, CD28, CD40, CD134, CD137, and NKG2D.

6. The virus of claim 1, wherein the cell surface molecule is CD3.

7. The virus of claim 1, wherein the oncolytic virus is vaccinia virus, adenovirus, Herpes simplex virus 1 (HSV1), myxoma virus, reovirus, poliovirus, vesicular stomatitis virus (VSV), measles virus (MV), or Newcastle disease virus (NDV).

8. A method of treating an individual for cancer, comprising the step of delivering to the individual a therapeutically effective amount of the virus of claim 1.

9. The method of claim 8, wherein the amount of the virus comprises 10⁵-10¹³ pfu of the virus.

10. The method of claim 8, further comprising the step of delivering to the individual an additional cancer therapy.

11. The method of claim 10, wherein the additional cancer therapy is surgery, radiation, chemotherapy, immunotherapy, hormone therapy, or a combination thereof.


SEQUENCE LISTING (7 sequences)

SEQ ID NO: 1 - protein, 502 aa, Artificial Sequence
  Synthetic Peptide
MDWIWRILFLVGAATGAHSQVQLLESGGGLVQPGGSLRLSCAASGFTFSSYTMSWVRQAPGQALEWMGTISSGGTYTYYPDSVKGRFTISRDNAKNSLYLQMNSLRAEDTAVYYCAREAIFTYWGRGTLVTSSGGGGSGGGGSGGGGSDIQLTQSPSSLSASVGDRVTITCKASQDINNYLSWYQQKPGQAPRLLIYRANRLVDGVPDRFSGSGYGTDFTLTINNIESEDAAYYFCLKYDVFPYTFGQGTKVEIKSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKS

SEQ ID NO: 2 - protein, 508 aa, Artificial Sequence
  Synthetic Peptide
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKALGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKS

SEQ ID NO: 3 - protein, 514 aa, Artificial Sequence
  Synthetic Peptide
MEFGLSWLFLVAILKGVQCSRDILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRADAAPTVSIFPGSGGGGSGGEVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKS

SEQ ID NO: 4 - protein, 381 aa, Artificial Sequence
  Synthetic Peptide
MDWIWRILFLVGAATGAHQVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKS

SEQ ID NO: 5 - protein, 508 aa, Artificial Sequence
  Synthetic Peptide
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKS

SEQ ID NO: 6 - protein, 968 aa, Artificial Sequence
  Synthetic Peptide
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKALFEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSASACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSGGGGSGGGGSGGGGSVDDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKS

SEQ ID NO: 7 - protein, 831 aa, Artificial Sequence
  Synthetic Peptide
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKALFEGAGGSGGSSGSDGASGSRVTAFSNMDDMLQKAHLVIEGTFIYLRDSTEFFIRVRDGWKKLQLGELIPIPADSPPPPALSSNPGAGGSGGSSGSDGASGSRASACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSGGGGSGGGGSGGGGSVDDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKS